(19) United States
(12) Patent Application Publication
(10) Pub. No.: US 2026/0365991 A1
(43) Pub. Date: Nov. 19, 2026

(54) COMPOUNDS FOR STABILIZING PEPTIDES IN BIOLOGICAL SAMPLES

(71) Applicant: Roche Diagnostics Operations, Inc., Indianapolis, IN (US)

(72) Inventors: Nicole Pirkl, Muenchen (DE); Martin Rempt, Penzberg (DE)

(21) Appl. No.: 19/658,367

(22) Filed: Apr. 24, 2026

Related U.S. Application Data
(63) Continuation of application No. PCT/EP2024/079880, filed on Oct. 23, 2024.

(30) Foreign Application Priority Data
Oct. 26, 2023 (EP) 23206165.5

(51) Int. Cl. C12N 5/00; C07K 14/595

(52) U.S. Cl. CPC C12N 5/526; C07K 14/595

(57) ABSTRACT

The present invention provides the use of a sulfone compound for stabilizing a peptide in a sample. Also provided is a corresponding method comprising adding a sulfone compound to a sample for stabilizing the peptide. Also provided is a vessel for collecting a sample comprising a peptide, wherein said vessel comprises a sulfone compound. Provided herein is further a composition comprising a peptide and a sulfone compound, a kit comprising a sulfone compound, a vessel of the invention or a composition of the invention. Finally, the present invention also relates to the use of a sulfone compound for inhibiting a protease.

### CROSS-REFERENCE TO RELATED APPLICATIONS

This application is a continuation of International PCT Application No. PCT/EP2024/079880 filed on Oct. 23, 2024, which claims priority to European Patent Application No. 23206165.5 filed on Oct. 26, 2023, the disclosures of which are hereby incorporated by reference in their entirety.

### SEQUENCE LISTING

This application incorporates by reference in its entirety the material in the ST.26 XML file titled ROCHE-291_Sequence_Listing.xml, which was created on Apr. 24, 2026 and is 20,222 bytes.

### FIELD OF THE INVENTION

The present invention relates to methods and reagents for stabilizing proteins and peptides such as gastrin peptides in biological samples, such as plasma or serum samples. In particular, the present invention provides for the use of a sulfone compound for stabilizing peptides and/or proteins in samples with proteolytic activity.

### BACKGROUND OF THE INVENTION

The stability of protein or peptide analytes in samples, especially in biological samples such as serum and plasma samples, can be hampered by analyte degradation. Especially, certain small peptides can be sensitive to degradation. Degradation of proteins or peptides in biological samples, which is typically caused by proteases comprised therein, can alter the results of assays detecting the amount of such proteins or peptides. Protein or peptide analytes with low stability in biological samples typically require that the sample is tested immediately, or is stored in a cold environment. However, even under cool storage the stability is often insufficient.

In certain instances, biological samples comprising protein or peptide prone to degradation in the biological sample are used as calibrator or control material for assays quantifying said protein or peptide. Accordingly, degradation of proteins and peptides in biological samples can also alter calibrator or control quality and shelf life.

Gastrin-17, which is a peptide hormone that is secreted by the G-cells of the antrum of the stomach, has been previously reported as a peptide analyte with limited stability in biological samples such as plasma and serum (see EP05739654).

EP05739654 has described the use of dimethylsulfoxid (DMSO) for the stabilization of gastrin and gastrin related peptides against degradation in a mammalian biological sample. DMSO has the disadvantage that it can be oxidized, i.e. is rather instable. Direct Protease inhibitors are complex molecules with partially permanent interaction with the peptide or other negative effects on immunoassay based diagnostics.

Thus, there is still a high need to provide improved means and methods for stabilizing peptides in samples, such as in particular blood samples like plasma or serum.

### SUMMARY OF THE INVENTION

The above-mentioned needs are addressed by the present invention. The present inventors have surprisingly found that sulfone compounds (e.g. of formula 3 as defined herein below) can significantly stabilize different peptides from degradation in mammalian biological samples, said peptides including but not limited to gastrin peptides, such as gastrin-17 peptides.

Surprisingly, the stabilizing effect of the sulfone compounds (e.g. according to formula 3) on the tested peptides and in particular the tested gastrin-17 peptide is superior to DMSO as previously used for stabilization of gastrin-17 peptides (see EP05739654). In particular, at longer incubation times (e.g. 23 or 24 hours) of such peptides in blood samples (e.g. serum), the peptide stability was increased using a sulfone compound (e.g. of formula 1 or formula 2) compared to similar concentrations of DMSO. It is also of note that sulfone compounds, especially of formula 1 or 2, are also less prone to oxidation, i.e. are more stable, as they build the next oxidation step after the sulfoxide in DMSO (Wasmus S and Vielstich W, Electrochimica Acta, Volume 38, Issues 2-3, February 1993, Pages 175-183). Accordingly in sum, herein provided are more stable (in particular less oxidation prone) peptide stabilizing agents with a surprisingly superior stabilizing activity vis-à-vis DMSO.

The present inventors also found for gastrin-17, as exemplary peptide, that a specific set of proteases (GluC, Chymotrypsin and/or Proline-endopeptidase) is responsible for the instability/degradation of the peptide in the employed blood samples (e.g. serum samples). Notably, these proteases all belong to the group of EC 3.4 (group of peptide bond hydrolases) and in particular the group of EC 3.4.21 (group of peptide bond hydrolases, which are serine proteases).

Consequently, there is ample evidence that the stabilizing effect observed in the blood samples as used in the appended Examples can be found also in other samples comprising one or more of these proteases, such as other biological samples comprising the respective proteases. Moreover, this data indicates that also other peptides that are degraded by these and/or other proteases can be protected from proteolytic degradation by using a sulfone compound. Accordingly, herein also provided is a novel approach to stabilize peptides and proteins against degradation by proteases, in particular proteases of EC 3.4 (group of peptide bond hydrolases) and in particular the group of EC 3.4.21 (group of peptide bond hydrolases, which are serine proteases). Such approach comprises adding a sulfone compound (e.g. of formula 3) to the sample.

In a first aspect, the present invention relates to the use of a sulfone compound (e.g. of formula 3 or in particular of formula 1 or 2) for stabilizing a peptide in a sample (e.g. a biological sample such as a blood sample).

In embodiments, the sulfone compound comprises less than 20 non-hydrogen atoms, even more preferably less than 10 non-hydrogen atoms. Non-hydrogen atoms means any chemical element other than hydrogen.

In another embodiment, the sulfone compound comprises less than 20 carbon atoms, even more preferably less than 10 carbon atoms.

In embodiments, the sulfone compound does not comprise any double and/or triple bonds between carbon atoms, i.e. does not comprise any carbon-carbon double and/or triple bonds.

In a specific embodiment, the sulfone compound does not comprise any double and triple bonds between carbon atoms and comprises less than 20 non-hydrogen atoms, even more preferably less than 10 non-hydrogen atoms.

In another specific embodiment, the sulfone compound does not comprise any double and triple bonds between carbon atoms and comprises less than 20 carbon atoms, even more preferably less than 10 carbon atoms.

In a particular embodiment, stabilizing a peptide means stabilizing a peptide from degradation, i.e. slowing down degradation of a peptide.

In a specific embodiment, the sulfone compound is of formula 3.

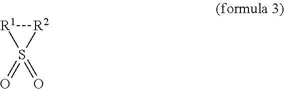

wherein R1 and R2 of formula 3 are either covalently linked via an R3 or not covalently linked,
wherein if R1 and R2 of formula 3 are covalently linked: i) R1 is selected from the group consisting of CH₂, CH—CH₃, NH and O, ii) R2 is selected from the group consisting of aliphatic linear or branched C1-C2, and iii) R3 is selected from the group consisting of aliphatic linear or branched C1-C4, CH—OR4 or C═O, wherein R4 is H or CH₃; or
wherein if R1 and R2 of formula 3 are not covalently linked: i) R1 is an aliphatic linear C1-C2, ii) R2 is an aliphatic linear C1-C2.

The dashed line between R1 and R2 in formula 3 indicates that R1 and R2 may either be covalently linked via R3 or not.

In embodiments, if R1 and R2 of formula 3 are covalently linked via R3, R3 is selected from aliphatic linear or branched C1-C3, CH—OR4 or C═O, wherein R4 is H or CH₃.

In preferred embodiments, Formula 3 does not comprise any carbon-carbon double and triple bonds. In other words, any of the aliphatic structures mentioned in the definition of formula 3 above are saturated aliphatic structures.

In an even more specific embodiment, the sulfone compound of the invention is selected from formula 2 or formula 1.

Formula 1 refers to the following compound(s):

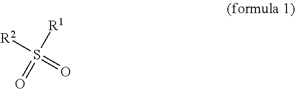

wherein R1 of formula 1 is an aliphatic linear C1-C2; and wherein R2 of formula 1 is an aliphatic linear C1-C2.

In embodiments, R1 of formula 1 is a saturated aliphatic linear C1-C2; and R2 of formula 1 is a saturated aliphatic linear C1-C2.

In embodiments R1 of formula 1 is CH3-CH2 or CH3.

In embodiments R1 of formula 1 is methyl or ethyl.

In embodiments R2 of formula 1 is CH3-CH2 or CH3.

In embodiments R2 of formula 1 is methyl or ethyl.

In embodiments R1 of formula 1 is methyl and R2 of formula 1 is ethyl. In embodiments R1 of formula 1 is methyl and R2 of formula 1 is methyl. In embodiments R1 of formula 1 is ethyl and R2 of formula 1 is ethyl.

Formula 2 as used herein refers to the following compound(s):

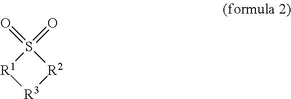

wherein R1 of formula 2 is selected from CH₂, CH—CH₃, NH and O,
wherein R2 of formula 2 is aliphatic linear or branched C1-C2,
wherein R3 of formula 2 is aliphatic linear or branched C1-C4, CH—OR4 or C═O, and wherein R4 is H or CH3.

In embodiments, R3 of formula 2 is aliphatic linear or branched C1-C3, CH—OR4 or C═O, and wherein R4 is H or CH3.

In embodiments, R2 of formula 2 is saturated aliphatic linear or branched C1-C2, and R3 of formula 2 is saturated aliphatic linear or branched C1-C4, CH—OR4 or C═O,
wherein R4 is H or CH₃.

In preferred embodiments R1 of formula 2 is CH₂, CH—CH₃ or O.

In preferred embodiments R2 of formula 2 is CH₂.

In preferred embodiments R3 of formula 2 is CH₂, CH₂—CH₂, CH2-CH2-CH2, CH₂—CHO—CH₂.

In a second aspect, the present invention relates to a method for stabilizing a peptide (e.g. from proteolytic degradation) in a sample (e.g. a biological sample such as a blood sample), said method comprising adding a sulfone compound to the sample comprising the peptide. In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bond between carbon atoms and comprises less than 20 non-hydrogen atoms, even more preferably less than 10 non-hydrogen atoms. In another specific embodiment, the sulfone compound does not comprise any double and triple bonds between carbon atoms and comprises less than 20 carbon atoms, even more preferably less than 10 carbon atoms.

In a specific embodiment, the sulfone compound is a compound of formula 3 as defined above. In an even more specific embodiment, the sulfone compound is a compound of formula 1 or 2 as defined above.

In a third aspect, the present invention provides a method for preprocessing a sample for the detection of a peptide in a sample (e.g. a biological sample such as a blood sample), wherein said method comprises stabilizing the peptide by adding a sulfone compound to the sample comprising the peptide. In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than 10 carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bond between carbon atoms and comprises less than 20 non-hydrogen atoms, even more preferably less than 10 non-hydrogen atoms. In another specific embodiment, the sulfone compound does not comprise any double and triple bonds between carbon atoms and comprises less than 20 carbon atoms, even more preferably less than 10 carbon atoms.

In a specific embodiment, the sulfone compound is a compound of formula 3 as defined above. In an even more specific embodiment, the sulfone compound is a compound of formula 1 or 2 as defined above.

In a fourth aspect, the present invention relates to a method for detecting a peptide in a sample (e.g. a biological sample such as a blood sample), wherein the method comprises (i) stabilizing the peptide in the sample by adding a sulfone compound to the sample comprising the peptide; and (ii) detecting the peptide in the sample. In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than 10 carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bond between carbon atoms and comprises less than 20 non-hydrogen atoms, even more preferably less than 10 non-hydrogen atoms. In another specific embodiment, the sulfone compound does not comprise any double and triple bonds between carbon atoms and comprises less than 20 carbon atoms, even more preferably less than 10 carbon atoms.

In a specific embodiment, the sulfone compound is a compound of formula 3 as defined above. In an even more specific embodiment, the sulfone compound is a compound of formula 1 or 2 as defined above.

In a fifth aspect, the present invention provides a vessel for collecting a sample (e.g. a blood sample), wherein said vessel comprises a sulfone compound.

In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than 10 carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bond between carbon atoms and comprises less than 20 non-hydrogen atoms, even more preferably less than 10 non-hydrogen atoms. In another specific embodiment, the sulfone compound does not comprise any double and triple bonds between carbon atoms and comprises less than 20 carbon atoms, even more preferably less than 10 carbon atoms.

In a specific embodiment, the sulfone compound is a compound of formula 3 as defined above. In an even more specific embodiment, the sulfone compound is a compound of formula 1 or 2 as defined above.

In a sixth aspect, the present invention relates to a composition (e.g. a calibrator or control material for a method for quantifying a peptide of interest) comprising a peptide (e.g. a gastrin peptide such as gastrin-17) and a sulfone compound.

In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than 10 carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bond between carbon atoms and comprises less than 20 non-hydrogen atoms, even more preferably less than 10 non-hydrogen atom. In another specific embodiment, the sulfone compound does not comprise any double and triple bonds between carbon atoms and comprises less than 20 carbon atoms, even more preferably less than 10 carbon atoms.

In a specific embodiment, the sulfone compound is a compound of formula 3 as defined above. In an even more specific embodiment, the sulfone compound is a compound of formula 1 or 2 as defined above.

According to a seventh aspect, the present invention provides a kit for (i) stabilizing a peptide in a sample, (ii) collecting a sample comprising a peptide, (iii) preprocessing a sample comprising a peptide for detection of the peptide or (iv) detecting a peptide in a sample, said kit comprising a sulfone compound, a composition of the sixth aspect of the invention and/or the vessel of the fifth aspect of the invention.

In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than 10 carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bond between carbon atoms and comprises less than 20 non-hydrogen atoms, even more preferably less than 10 non-hydrogen atoms. In another specific embodiment, the sulfone compound does not comprise any double and triple bonds between carbon atoms and comprises less than 20 carbon atoms, even more preferably less than 10 carbon atoms.

In a specific embodiment, the sulfone compound is a compound of formula 3 as defined above. In an even more specific embodiment, the sulfone compound is a compound of formula 1 or 2 as defined above.

In embodiments, the composition is a calibrator or control material for a method for detecting a peptide of interest. For example, the composition may comprise a predefined concentration of a peptide of interest (e.g. a gastrin peptide such as gastrin-17) and a sulfone compound.

In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than 10 carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bond between carbon atoms and comprises less than 20 non-hydrogen atoms, even more preferably less than 10 non-hydrogen atoms. In another specific embodiment, the sulfone compound does not comprise any double and triple bonds between carbon atoms and comprises less than 20 carbon atoms, even more preferably less than 10 carbon atoms. In a specific embodiment, the sulfone compound is a compound of formula 3 as defined above. In an even more specific embodiment, the sulfone compound is a compound of formula 1 or 2 as defined above.

In an eighth aspect, the present invention relates to the use of a sulfone compound for inhibiting a protease.

In embodiments, the sulfone compound comprises less than 20 non-hydrogen atoms, even more preferably less than 10 non-hydrogen atoms. Non-hydrogen atoms means any chemical element other than hydrogen. In another embodiment, the sulfone compound comprises less than 20 carbon atoms, even more preferably less than 10 carbon atoms. In embodiments, the sulfone compound does not comprise any double and/or triple bonds between carbon atoms, i.e. does not comprise any carbon-carbon double and/or triple bonds. In a specific embodiment, the sulfone compound does not comprise any double and triple bonds between carbon atoms and comprises less than 20 non-hydrogen atoms, even more preferably less than 10 non-hydrogen atoms. In another specific embodiment, the sulfone compound does not comprise any double and triple bonds between carbon atoms and comprises less than 20 carbon atoms, even more preferably less than 10 carbon atoms.

In a specific embodiment, the sulfone compound is a compound of formula 3 as defined above. In an even more specific embodiment, the sulfone compound is a compound of formula 1 or 2 as defined above. The one or more protease may be selected from but are not limited to the group of EC 3.4 (group of peptide bond hydrolases) and the peptide is a cleavage target of such one or more protease. In an even more preferred embodiment, the protease is selected from the group of EC 3.4.21 (group of peptide bond hydrolases, which are serine proteases). In an even more preferred embodiment, the protease is selected from the group of: a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26).

### DETAILED DESCRIPTION OF THE INVENTION

In a first aspect, the present invention relates to the in vitro use of a sulfone compound for stabilizing a peptide in a sample (e.g. a biological sample such as a blood sample).

Preferably, the stabilization is a stabilization from degradation, even more preferably proteolytic degradation (i.e. cleavage of the peptide by one or more proteases).

In embodiments, the sulfone compound has a logP value of lower than 0.2. In embodiments, the sulfone compound has a logP value of lower than −0.13 with an error of +/−0.30.

Sulfone compounds with correspondingly low logP values have the advantage of being suitably water soluble to be used in aqueous samples such as body fluids, in particular blood samples. As demonstrated in the appended Examples the sulfone compounds have calculated logP values in this range.

The partition coefficient, abbreviated P, is defined as a particular ratio of the concentrations of a solute between the two solvents (a biphase of liquid phases), specifically for un-ionized solutes, and the logarithm of the ratio is thus log P. When one of the solvents is water and the other is a non-polar solvent such as n-octanol, then the log P value is a measure of lipophilicity or hydrophobicity. The defined precedent is for the lipophilic and hydrophilic phase types to always be in the numerator and denominator respectively. In the context of the present invention the biphasic system for defining P and logP is n-octanol and water, i.e. the logP value is logP_{Octanol/water}. The logP value may be experimentally determined or calculated. Preferably the logP refers to the calculated logP value. The logP is preferably calculated using ACD/CehmSketch 2018.1 (File version C50H41).

In embodiments, the sulfone compound comprises less than 20 non-hydrogen atoms, even more preferably less than 10 non-hydrogen atoms. Non-hydrogen atoms means any chemical element other than hydrogen. Smaller sulfone compounds have the advantage that they exhibit a better atomic balance, thereby providing the active element (sulfur in the oxidation state+II) without disruptive shielding effects or opportunities for side reactions or instabilities.

In another embodiment, the sulfone compound comprises less than 20 carbon atoms, even more preferably less than 10 carbon atoms.

In embodiments, the sulfone compound does not comprise any double and/or triple bond between carbon atoms; i.e. does not comprise any double and/or triple carbon-carbon bonds. In other words, the sulfone compound does not comprise any non-saturated carbon-carbon bonds. As carbon-carbon double and triple bonds are rather reactive, especially in connection with compounds comprising heteroatoms, the absence of such double bonds is preferred to avoid any undesired chemical reactions of the sulfone compound interfering with its peptide protection capacity.

In a specific embodiment, the sulfone compound does not comprise any double and triple bond between carbon atoms and comprises less than 20 non-hydrogen atoms, even more preferably less than 10 non-hydrogen atoms. In another specific embodiment, the sulfone compound does not comprise any double and triple bond between carbon atoms and comprises less than 20 carbon atoms, even more preferably less than 10 carbon atoms.

In a specific embodiment, the sulfone compound is of formula 3.

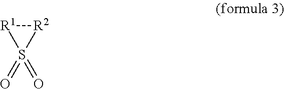

wherein R1 and R2 of formula 3 are either covalently linked via a R3 or not covalently linked,
wherein if R1 and R2 of formula 3 are covalently linked: i) R1 is selected from the group consisting of CH₂, CH—CH₃, NH and O, ii) R2 is selected from the group consisting of aliphatic linear or branched C1-C2, and iii) R3 is selected from the group consisting of aliphatic linear or branched C1-C4, CH—OR4 or C═O, wherein R4 is H or CH₃; or
wherein if R1 and R2 of formula 3 are not covalently linked: i) R1 is an aliphatic linear C1-C2, ii) R2 is an aliphatic linear C1-C2.

The dashed line between R1 and R2 in formula 3 indicates that these may either be covalently linked via R3 or not.

In embodiments, if R1 and R2 of formula 3 are covalently linked R 3 is selected from aliphatic linear or branched C1-C3, CH—OR4 or C═O, wherein R4 is H or CH₃.

In preferred embodiments, Formula 3 does not comprise any carbon-carbon double and triple bonds. In other words, any of the aliphatic structures mentioned in the definition of formula 3 above are saturated aliphatic structures.

In an even more specific embodiment, the sulfone compound is selected from formula 2 or formula 1.

Formula 1 refers to the following compound(s):

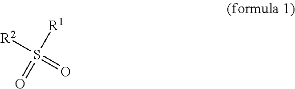

wherein R1 of formula 1 is an aliphatic linear C1-C2; and
wherein R2 of formula 1 is an aliphatic linear C1-C2.

In embodiments, R1 of formula 1 is a saturated aliphatic linear C1-C2; and R2 of formula 1 is a saturated aliphatic linear C1-C2. In embodiments R1 of formula 1 is CH3-CH2 or CH3.

In embodiments R1 of formula 1 is methyl or ethyl.

In embodiments R2 of formula 1 is CH3-CH2 or CH3.

In embodiments R2 of formula 1 is methyl or ethyl.

In embodiments R1 of formula 1 is methyl and R2 of formula 1 is ethyl. In embodiments R1 of formula 1 is methyl and R2 of formula 1 is methyl. In embodiments R1 of formula 1 is ethyl and R2 of formula 1 is ethyl.

Formula 2 as used herein refers to the following compound(s):

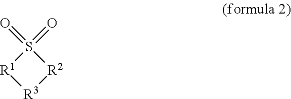

wherein R1 of formula 2 is selected from CH₂, CH—CH₃, NH and O,
wherein R2 of formula 2 is aliphatic linear or branched C1-C2,
wherein R3 of formula 2 is aliphatic linear or branched C1-C4, CH—OR4 or C═O, and wherein R4 is H or CH₃.

In embodiments, R1 of formula 2 is selected from CH₂, CH—CH₃, NH and O; R2 of formula 2 is saturated aliphatic linear or branched C1-C2; and R3 of formula 2 is saturated aliphatic linear or branched C1-C4, CH—OR4 or C═O, wherein R4 is H or CH₃.

In preferred embodiments R1 of formula 2 is CH₂, CH—CH₃ or O.

In preferred embodiments R2 of formula 2 is CH₂.

In preferred embodiments R3 of formula 2 is CH₂, CH₂—CH₂, or CH₂—CHO—CH₂.

In specific embodiments, the sulfone compound is selected from the group consisting of: sulfolane (CAS: 126-33-0); trimethylene sulfone (CAS: 5687-92-3); 1,1-dioxoisothiazolidine (CAS: 5908-62-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4). As demonstrated in the appended Examples, all these compounds have the ability to stabilize peptides (e.g. gastrin-17) in samples (e.g. blood samples such as plasma or serum). Furthermore, all these substances are more stable against oxidation than DMSO (Wasmus S and Vielstich W, Electrochimica Acta, Volume 38, Issues 2-3, February 1993, Pages 175-183).

In specific embodiments, the sulfone compound is selected from the group consisting of: sulfolane (CAS: 126-33-0); trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4). As demonstrated in the appended Examples, all these compounds had an ability to stabilize peptides (e.g. gastrin-17) in samples (e.g. blood samples such as plasma or serum) that is superior to the stabilizing ability of DMSO.

In specific embodiments, the sulfone compound is sulfolane (CAS: 126-33-0). Sulfolane is industrially produced in large amounts, i.e. is easily available in large amounts.

In specific embodiments, the sulfone compound is trimethylene sulfone (CAS: 5687-92-3).

In specific embodiments, the sulfone compound is 1,4-butanesultone (CAS: 1633-83-6).

In specific embodiments, the sulfone compound is tetrahydro-2H-thiopyran-4-ol 1,1-dioxide (CAS: 194152-05-1).

In specific embodiments, the sulfone compound is methyltetrahydrothiophene 1,1-dioxide (CAS: 1003-46-9).

In specific embodiments, the sulfone compound is ethylmethylsulfone (CAS: 594-43-4).

The term peptide as used herein includes any molecules consisting of or essentially consisting of amino acids. The term also includes modified peptides. A peptide as used herein can be of any length of amino acids and includes proteins (e.g. preprogastrin or progastrin). The terms peptide and protein are in principle used interchangeably herein. In embodiments, peptides comprise at most 40 amino acids and proteins comprise at least 41 amino acids.

A peptide or a protein as employed in the present invention may be a peptide or protein that is a naturally occurring in a sample. Alternatively, the peptide or protein may be synthetic or recombinantly expressed.

The peptide to be stabilized may be different in length. In embodiments, the peptide comprises at least 4, at least 5, at least 6, at least 7 or at least 8 amino acids. In embodiments, the peptide comprises at most 22, at most 30, at most 34, at most 35, at most 36, at most 37, at most 38, at most 39 or at most 40 amino acids.

In embodiments, the peptide is of 4 to 40 amino acids in length. In embodiments, the peptide is of 6 to 34 amino acids in length. In embodiments, the peptide is of 8 to 22 amino acids in length. In a specific embodiment, the peptide is of 4 to 22 amino acids in length. In an embodiment, the peptide is of 17 to 34 amino acids in length.

As found in the appended examples, the sulfone compounds such as of formula 3, formula 1 or formula 2 protect gastrin peptides from degradation in a sample. In particular, the peptides are protected from degradation via GluC (EC 3.4.21.19), Chymotrypsin (EC 3.4.21.1) and/or Proline-endopeptidase (EC 3.4.21.26), which all belong to the group of peptide bond hydrolases (EC 3.4) and serine proteases (EC 3.4.21).

Thus, in a preferred embodiment, the peptide is a peptide comprising one or more recognition sites for a protease. In an even more preferred embodiment, the peptide is a peptide comprising one or more recognition sites for a protease selected from the group of EC 3.4 (group of peptide bond hydrolases). In an even more preferred embodiment, the peptide is a peptide comprising one or more recognition sites for a protease selected from the group of EC 3.4.21 (group of peptide bond hydrolases, which are serine proteases). In an even more preferred embodiment, the peptide is a peptide comprising one or more recognition sites for a protease selected from the group of: a GluC protease (i.e. an enzyme of EC 3.4.21.19), Chymotrypsin (i.e. an enzyme of EC 3.4.21.1) and Proline-endopeptidase (i.e. an enzyme of EC 3.4.21.26).

In preferred embodiments, the peptide of the invention is a gastrin peptide, e.g. a gastrin-17 peptide or a gastrin-34 peptide. In an even more preferred embodiment, the peptide is a gastrin-17 peptide.

Gastrin is a linear peptide hormone produced by G cells of the duodenum and in the pyloric antrum of the stomach of humans. It is secreted into the bloodstream. The encoded polypeptide is preprogastrin, which is cleaved by enzymes in posttranslational modification to produce progastrin (an intermediate, inactive precursor) and then gastrin in various forms, primarily the following three: gastrin-34, gastrin-17, gastrin-14. Also, pentagastrin is an artificially synthesized, five amino acid sequence identical to the last five amino acid sequence at the C-terminus end of gastrin. The numbers refer to the amino acid count.

The terms “gastrin peptide” or “gastrin peptides” as used herein relate to any peptide with a length of minimum 5 amino acids derived from preprogastrin (SEQ ID NO: 1). The sequences of preprogastrin and corresponding cleavage sites resulting in gastrin peptides are summarized in the UniProt entry P01350 (version 195). In particular, “gastrin peptide” or “gastrin peptides” include all naturally occurring gastrin peptides including but not limited to preprogastrin, progastrin, gastrin-34, gastrin-17, gastrin-14 and gastrin-6. In one embodiment, the gastrin peptide is selected from the group consisting of: preprogastrin, progastrin, gastrin-34, gastrin-17, gastrin-14. In a specific embodiment, the gastrin peptide is selected from the group consisting of: gastrin-34 and gastrin-17.

Preprogastrin (SEQ ID NO: 1 shows human preprogastrin) is translated at the endoplasmic reticulum, where an N-terminal signaling sequence is cleaved from the peptide to yield progastrin. Progastrin (SEQ ID NO: 2 shows human Progastrin) then passes through the Golgi complex and the trans-Golgi network, where it can be sulfated on a tyrosine corresponding to the tyrosine residue in position 86 of preprogastrin (which becomes part of Gastrin-17 and Gastrin-34). The sulfation is not complete, i.e. gastrin peptides with and without sulfate on the Tyr corresponding to Tyr-86 or preprogastrin exist. Progastrin is further packaged into secretory vesicles, in which the dibasic sites corresponding to Arg36-Arg37 and Arg73-Arg74 are cleaved on the C-terminal side by the prohormone convertases PC1/3 and PC2, and the remaining C-terminal basic residues Arg73-Arg74 are removed by carboxypeptidase E to give the peptide gastrin-34-Gly (i.e. gastrin-34 with a C-terminal glycine). In the stomach, Gastrin-34-Gly is further processed by PAM enzyme to give the mature gastrin-34, which has a C-terminal amide group instead of the glycine. Mature gastrin-34 can be further processed to mature gastrin-17 by enzymatic cleavage and conversion of the N-terminal glutamine to pyro-glutamate. Alternatively, in the colon, gastrin-34-Gly can be processed to gastrin-17-Gly, i.e. having an N-terminal pyro-glutamate residue and a C-terminal glycine. The processing steps of preprogastrin including the different gastrin forms are for examples described in Copps J et al. (Protein Pept Lett. 2009; 16(12): 1504-1518. doi: 10.2174/092986609789839269).

The term gastrin-17 or gastrin-17 peptide as referred to herein relates to any types of glycin-17 peptides, i.e. gastrin-17-Gly, mature gastrin-17 (with C-terminal amide group instead of Gly in gastrin-17-Gly) both either sulfated or not sulfated at tyrosine in position 12 of the gastrin primary sequence. In other word, gastrin-17 as used herein includes and preferably relates to gastrin-17-Gly (SEQ ID NO: 3) and mature gastrin-17 (SEQ ID NO: 4). Both gastrin-17-Gly and mature gastrin-17 can be sulfated or not at the tyrosine at position 12. An optional further modification to any such forms of gastrin-17 are oxidation of the methionine at position 15, which can spontaneously occur, in particular in ex vivo samples.

Gastrin-34 or gastrin-34 peptide as used herein includes or preferably refers to gastrin 34-Gly (SEQ ID NO: 5) and mature gastrin-34 (with C-terminal amide group; SEQ ID NO: 6). Both gastrin-34-Gly and mature gastrin-34 can be sulfated or not at the tyrosine at position 29 (corresponding to position 12 of Gastrin-17). An optional further modification to any such forms of gastrin-34 are oxidation of the methionine at position 32 (corresponding to position 15 of gastrin-17), which can spontaneously occur especially ex vivo.

Gastrin-14 as used herein refers to a 14 amino acid peptide. The amino acid sequence of human gastrin-14 is depicted in SEQ ID NO: 7

As mentioned above the stabilization of the peptide is preferably a stabilization from proteolytic degradation. Said proteolytic degradation may be caused by proteases, e.g. that are naturally occurring in the sample (e.g. a blood sample such as serum and/or plasma).

Accordingly, in embodiments, the sample (e.g. a blood sample such as serum and/or plasma) may comprise one or more protease and the peptide to be stabilized comprises one or more recognition site for said one or more protease (i.e. is a cleavage substrate of the one or more protease comprised in the sample.

As illustrated in the appended Examples, the inventors have found that the proteases GluC (EC 3.4.21.19), Chymotrypsin (EC 3.4.21.1) and/or Proline-endopeptidase (EC 3.4.21.26) caused degradation of gastrin-17 (which comprises corresponding recognition sites). These proteases all belong to the group of peptide bond hydrolases (EC 3.4) and serine proteases (EC 3.4.21), i.e. strongly suggesting that sulfolanes (e.g. of formula 3 or in particular of formula 2 or 1) protect peptides and proteins from degradation by these enzyme classes.

In preferred embodiments, the sample (e.g. a blood sample such as serum and/or plasma) comprise one or more protease and the peptide or protein to be stabilized comprises one or more recognition site for said one or more protease, wherein the one or more protease is selected from the group of EC 3.4 (e.g. group of peptide bond hydrolases).

In even more preferred embodiments, the sample (e.g. a blood sample such as serum and/or plasma) comprise one or more protease and the peptide or protein to be stabilized comprises one or more recognition site for said one or more protease, wherein the one or more protease is selected from EC 3.4.21 (e.g. group of peptide bond hydrolases, which are serine proteases).

In even more preferred embodiments, the sample (e.g. a blood sample such as serum and/or plasma) comprise one or more protease and the peptide or protein to be stabilized comprises one or more recognition site for said one or more protease, wherein the one or more protease is selected from the group consisting of a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26).

The sample as used in the context of the present invention can be any sample comprising the peptide to be stabilized (can be naturally present or spiked in) and one or more protease capable of cleaving the peptide.

In a preferred embodiment, the sample is liquid. In an even more preferred embodiment, the sample is a body fluid. A body fluid may, for example be selected from blood, urine, cerebrospinal fluid (CSF) or saliva.

In a particularly preferred embodiment, the sample is a blood sample, such as a venous blood sample or a capillary blood sample. In an even more preferred embodiment, the sample is a serum or a plasma sample.

In embodiments, the sample is an animal sample, preferably a mammalian sample, even more preferably a human or horse sample. In a particularly preferred embodiment, the sample is a human sample.

In embodiments, the sample may be a peptide mixture, e.g. a mixture of peptides for use in mass spectrometry. Such mixture may be commercially purchased or generated by protease digest of a protein sample.

In embodiments, the sample is a sample comprising (i) a protease selected from a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26); and (ii) a peptide (e.g. a gastrin peptide such as gastrin-17) having at a recognition site for the protease (i.e. peoptide being a cleavage substrate of the protease in this sample). Optionally the sample is a blood sample (e.g. serum or plasma). Blood samples are known to comprise those proteases and corresponding substrate peptides such as gastrin peptides (e.g. gastrin-17).

In embodiments, the use of the sulfone compounds as stabilizing agent is in the context of a temperature in the range of 0 °C to 30 °C, preferably 4 °C to 28 °C and most preferably °C to 25 °C. In other words, the use may comprise incubating the peptide in the sample (e.g. a biological sample such as a blood sample) at temperature of 0 °C to 30 °C, preferably 4 °C to 28 °C and most preferably 15 °C to 25 °C. In embodiments, the use is in a temperature scale of −80 °C to 27 °C. In embodiments, the use is in a temperature scale of −20 °C to 27 °C. In embodiments, the use is in a temperature scale of 4 °C and 27 °C.

In embodiments, the peptide is incubated in the presence of the sulfone compound in the sample (e.g. a biological sample such as a blood sample) for more than 5 hours, preferably more than 8 hours and even more preferably more than 23 hours or more than 24 hours. In embodiments, the incubation of the peptide in the sample is less than 8 weeks. In embodiments, the incubation of the peptide in the sample is less than 2 weeks. In embodiments, the incubation of the peptide in the sample is less than 7 days. In embodiments, the incubation of the peptide in the sample is less than 3 days.

The sulfone compound (e.g. of formula 3 or more particularly of formula 1 or 2) is typically added to the sample. Accordingly, in embodiments, the use of the first aspect of the invention comprises adding a sulfolane compound to the sample. The sample may then be incubated (e.g. under the temperatures and/or for the times indicated above) in the presence of the sulfone compound.

As evidenced by the appended Examples a sulfolane compound, such as of formula 3 (e.g. of formula 1 or 2) can be present in different concentrations to achieve the technical effect of peptide stabilization.

In embodiments, the concentration of the sulfone compound in the final incubation mixture (i.e. the sample comprising the peptide and the added compound of formula 1 or 2) is at least 1% (v/v), in particular at least 1.25% (v/v), in particular at least 2% (v/v), in particular at least 2.5% (v/v) and in particular at least 5% (v/v). In embodiments, the concentration of the compound of the sulfone compound in the final incubation mixture (i.e. the sample comprising the peptide and the added sulfone compound) is at most 5% (v/v), in particular at most 10% (v/v) and in particular at most 20% (v/v).

The sulfone compound is preferably selected such that the stabilization of the peptide in the sample is higher than with the equimolar concentration of DMSO in the same sample. Preferably, the stability is assessed by measuring the peptide level at the time point of mixing in the sulfone compound or DMSO and the peptide level after a defined time, the time being preferably 23 h or 24 h. Then the levels are compared as to assess the stability.

The sulfone compound of the invention is not DMSO.

In a particular embodiment of the first aspect of the invention provided is the use of a compound for stabilizing a peptide in a sample from proteolytic degradation by one or more of the proteases selected from the group consisting of GluC, Chymotrypsin and Proline-endopeptidase, wherein the sample comprises said one or more proteases and the peptide is cleaved by said one or more proteases (i.e. comprises a recognition motif for said one or more proteases), wherein the compound is a compound of formula 3 (e.g. of formula 1 or 2).

In a particular embodiment of the first aspect of the invention provided is the use of a compound for stabilizing a peptide in a sample from proteolytic degradation by one or more of the proteases selected from the group consisting of GluC, Chymotrypsin and Proline-endopeptidase, wherein the sample comprises said one or more proteases and the peptide is cleaved by said one or more proteases (i.e. comprises a recognition motif for said one or more proteases), wherein the compound is a compound selected from the group consisting of sulfolane (CAS: 126-33-0); trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4).

In a particular embodiment of the first aspect of the invention provided is the use of a compound for stabilizing a gastrin peptide in a sample from proteolytic degradation, e.g. by one or more of the proteases selected from the group consisting of GluC, Chymotrypsin and Proline-endopeptidase, wherein the sample comprises said one or more proteases, wherein the compound is a compound of formula 1 or 2.

In a particular embodiment of the first aspect of the invention provided is the use of a compound for stabilizing a gastrin peptide in a sample from proteolytic degradation, e.g. by one or more of the proteases selected from the group consisting of GluC, Chymotrypsin and Proline-endopeptidase, wherein the sample comprises said one or more proteases, wherein the compound is a compound selected from the group consisting of sulfolane (CAS: 126-33-0); trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4).

In a second aspect, the present invention relates to a method for stabilizing a peptide (e.g. against degradation) in a sample (e.g. a biological sample such as a blood sample), said method comprising adding a sulfone compound to the sample comprising the peptide.

What has been said elsewhere herein, especially any embodiments and definitions mentioned herein elsewhere with respect to other aspects (e.g. with respect to the use of the first aspect) apply to this second aspect of the invention mutatis mutandis.

In particular all embodiments of the use according to the first aspect apply mutatis mutandis.

In a particular embodiment of the second aspect of the invention provided is a method for stabilizing a peptide in a sample from proteolytic degradation by one or more of the proteases (e.g. selected from the group consisting of GluC, Chymotrypsin and Proline-endopeptidase), wherein the sample comprises said one or more proteases and the peptide is cleaved by said one or more proteases (i.e. comprises a recognition motif for said one or more proteases), said method comprising adding a compound to the sample, wherein the compound is a compound of formula 3 (e.g. of formula 1 or 2).

In a particular embodiment of the second aspect of the invention provided is a method for stabilizing a peptide in a sample from proteolytic degradation by one or more of the proteases (e.g. selected from the group consisting of GluC, Chymotrypsin and Proline-endopeptidase), wherein the sample comprises said one or more proteases and the peptide is cleaved by said one or more proteases (i.e. comprises a recognition motif for said one or more proteases), said method comprising adding a compound to the sample, wherein the compound is a compound selected from the group consisting of sulfolane (CAS: 126-33-0); trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4).

In a particular embodiment of the second aspect of the invention provided is a method for stabilizing a gastrin peptide in a sample from proteolytic degradation by one or more of the proteases (e.g. selected from the group consisting of GluC, Chymotrypsin and Proline-endopeptidase), said method comprising adding a compound to the sample, wherein the sample comprises said one or more proteases, wherein the compound is a sulfone compound (e.g. of formula 3, more specifically of formula 1 or 2).

In a particular embodiment of the second aspect of the invention provided is a method for stabilizing a gastrin peptide in a sample from proteolytic degradation (e.g. by one or more of the proteases selected from the group consisting of GluC, Chymotrypsin and Proline-endopeptidase), wherein the sample comprises said one or more proteases, said method comprising adding a compound to the sample, wherein the compound is a compound selected from the group consisting of sulfolane (CAS: 126-33-0); trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4).

In a third aspect, the present invention provides a method for preprocessing a sample for the detection of a peptide in a sample (e.g. a biological sample such as a blood sample), wherein said method comprises stabilizing the peptide by a method for stabilizing a peptide as defined herein in the context of the second aspect of the invention and any embodiments thereof.

What has been said elsewhere herein, especially any embodiments and definitions mentioned herein elsewhere with respect to other aspects (e.g. with respect to the first and second aspect) applies to this third aspect of the invention mutatis mutandis.

What has been said in context of the first and/or second aspect of the invention herein above with respect to the sample, the peptide and the stabilization method using a sulfone compound applies mutatis mutandis.

In a fourth aspect, the present invention relates to a method for detecting a peptide in a sample (e.g. a biological sample such as a blood sample), wherein the method comprises (i) stabilizing the peptide in the sample adding a sulfone compound (e.g. a compound of formula 3, such as a compound of formula 1 or 2) to the sample comprising the peptide; and (ii) detecting the peptide in the sample.

What has been said elsewhere herein, especially any embodiments and definitions mentioned herein elsewhere with respect to other aspects (e.g. with respect to the first, second and third aspect) applies to this fourth aspect of the invention mutatis mutandis.

What has been said herein elsewhere about the method for stabilizing a peptide according the second aspect and the corresponding use of the first aspect applies to step (i) of the method of the fourth aspect of the invention mutatis mutandis.

What has been said with respect to the sulfone compound, the peptide and the sample in context of the first, second and third aspect applies mutatis mutandis.

Stabilizing the peptide may be started directly after the sample has been obtained (e.g. directly after a blood draw) by adding the sulfone compound to the sample.

Detecting the sample can be achieved by different methods including but not limited to: immunoassays using a detectable label such as electrochemiluminescence label (e.g. Ruthenium), fluorescence label, or an enzymatic label, or mass spectrometry detection method.

In an exemplary embodiment, the detection may involve contacting the sample with a first binding agent specifically binding the peptide and detecting the presence and/or amount of the peptide by detecting the formation of a complex between the first binding agent with the peptide. An exemplary binding agent is an antibody.

In a fifth aspect, the present invention provides a device or vessel for collecting a sample (e.g. a blood sample), wherein said vessel comprises a sulfone compound (e.g. a compound of formula 3 as defined herein above, such as a compound of formula 1 or 2 as defined herein above).

What has been said elsewhere herein, especially any embodiments and definitions mentioned herein elsewhere with respect to other aspects (e.g. with respect to the first, second, third and fourth aspect) applies to this fifth aspect of the invention mutatis mutandis.

In embodiments, the sulfone compound comprises less than 20 non-hydrogen atoms, even more preferably less than 10 non-hydrogen atoms. Non-hydrogen atoms means any chemical element other than hydrogen.

In another embodiment, the sulfone compound comprises less than 20 carbon atoms, even more preferably less than 10 carbon atoms.

In embodiments, the sulfone compound does not comprise any double and/or triple bonds between carbon atoms, i.e. does not comprise any carbon-carbon double and/or triple bonds.

In a specific embodiment, the sulfone compound does not comprise any double and triple bonds between carbon atoms and comprises less than 20 non-hydrogen atoms, even more preferably less than 10 non-hydrogen atoms.

In another specific embodiment, the sulfone compound does not comprise any double and triple bonds between carbon atoms and comprises less than 20 carbon atoms, even more preferably less than 10 carbon atoms. In embodiments, the sulfone compound has a logP value of lower than 0.2. In embodiments, the suldone compound has a logP value of lower than −0.13 with an error of +/−0.30.

Sulfone compounds with correspondingly low logP values have the advantage of being suitably water soluble to be used in aqueous samples such as body fluids, in particular blood samples. As demonstrated in the appended Examples the sulfone compounds have calculated logP values in this range.

The partition coefficient, abbreviated P, is defined as a particular ratio of the concentrations of a solute between the two solvents (a biphase of liquid phases), specifically for un-ionized solutes, and the logarithm of the ratio is thus log P. When one of the solvents is water and the other is a non-polar solvent such as n-octanol, then the log P value is a measure of lipophilicity or hydrophobicity. The defined precedent is for the lipophilic and hydrophilic phase types to always be in the numerator and denominator respectively. In the context of the present invention the biphasic system for defining P and logP is n-octanol and water, i.e. the logP value is logP_{Octanol/water}. The logP value may be experimentally determined or calculated. Preferably the logP refers to the calculated logP value. The logP is preferably calculated using ACD/CehmSketch 2018.1 (File version C50H41).

In a specific embodiment, the sulfone compound is of formula 3.

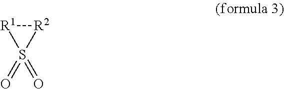

wherein R1 and R2 of formula 3 are either covalently linked via a R3 or not covalently linked,
wherein if R1 and R2 of formula 3 are covalently linked: i) R1 is selected from the group consisting of CH₂, CH—CH₃, NH and O, ii) R2 is selected from the group consisting of aliphatic linear or branched C1-C2, and iii) R3 is selected from the group consisting of aliphatic linear or branched C1-C4, CH—OR4 or C═O, wherein R4 is H or CH₃; or
wherein if R1 and R2 of formula 3 are not covalently linked: i) R1 is an aliphatic linear C1-C2, ii) R2 is an aliphatic linear C1-C2.

The dashed line between R1 and R2 in formula 3 indicates that these may either be covalently linked via R3 or not.

In embodiments, if R1 and R2 of formula 3 are covalently linked R 3 is selected from aliphatic linear or branched C1-C3, CH—OR4 or C═O, wherein R4 is H or CH₃.

In preferred embodiments, Formula 3 does not comprise any carbon-carbon double and triple bonds. In other words, any of the aliphatic structures mentioned in the definition of formula 3 above are saturated aliphatic structures.

In an even more specific embodiment, the sulfone compound is selected from formula 2 or formula 1.

Formula 1 refers to the following compound(s):

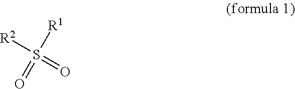

wherein R1 of formula 1 is an aliphatic linear C1-C2; and
wherein R2 of formula 1 is an aliphatic linear C1-C2.

In embodiments, R1 of formula 1 is a saturated aliphatic linear C1-C2; and R2 of formula 1 is a saturated aliphatic linear C1-C2.

In embodiments R1 of formula 1 is CH3-CH2 or CH3.

In embodiments R1 of formula 1 is methyl or ethyl.

In embodiments R2 of formula 1 is CH3-CH2 or CH3.

In embodiments R2 of formula 1 is methyl or ethyl.

In embodiments R1 of formula 1 is methyl and R2 of formula 1 is ethyl. In embodiments R1 of formula 1 is methyl and R2 of formula 1 is methyl. In embodiments R1 of formula 1 is ethyl and R2 of formula 1 is ethyl.

Formula 2 as used herein refers to the following compound(s):

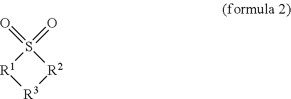

wherein R1 of formula 2 is selected from CH₂, CH—CH₃, NH and O,
wherein R2 of formula 2 is aliphatic linear or branched C1-C2,
wherein R3 of formula 2 is aliphatic linear or branched C1-C4, CH—OR4 or C═O, and wherein R4 is H or CH₃.

In embodiments, R1 of formula 2 is selected from CH₂, CH—CH₃, NH and O; R2 of formula 2 is saturated aliphatic linear or branched C1-C2; and R3 of formula 2 is saturated aliphatic linear or branched C1-C4, CH—OR4 or C═O, wherein R4 is H or CH₃.

In preferred embodiments R1 of formula 2 is CH₂, CH—CH₃ or O.

In preferred embodiments R2 of formula 2 is CH₂.

In preferred embodiments R3 of formula 2 is CH₂, CH₂—CH₂, CH₂—CHO—CH₂.

In specific embodiments, the sulfone compound is selected from the group consisting of: sulfolane (CAS: 126-33-0); trimethylene sulfone (CAS: 5687-92-3); 1,1-dioxoisothiazolidine (CAS: 5908-62-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4). As demonstrated in the appended Examples, all these compounds have the ability to stabilize peptides (e.g. gastrin-17) in samples (e.g. blood samples such as plasma or serum). Furthermore, all these substances are more stable against oxidation than DMSO.

In specific embodiments, the sulfone compound is selected from the group consisting of: sulfolane (CAS: 126-33-0); trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4). As demonstrated in the appended Examples, all these compounds had an ability to stabilize peptides (e.g. gastrin-17) in samples (e.g. blood samples such as plasma or serum) that is superior to the stabilizing ability of DMSO.

In specific embodiments, the sulfone compound is sulfolane (CAS: 126-33-0). Sulfolane is industrially produced in large amounts, i.e. is easily available in large amounts.

In specific embodiments, the sulfone compound is trimethylene sulfone (CAS: 5687-92-3).

In specific embodiments, the sulfone compound is 1,4-butanesultone (CAS: 1633-83-6).

In specific embodiments, the sulfone compound is tetrahydro-2H-thiopyran-4-ol 1,1-dioxide (CAS: 194152-05-1).

In specific embodiments, the sulfone compound is methyltetrahydrothiophene 1,1-dioxide (CAS: 1003-46-9).

In specific embodiments, the sulfone compound is ethylmethylsulfone (CAS: 594-43-4).

The vessel or device is a vessel or device suitable for collecting a sample. The sample is preferably a sample as defined in the context of the first aspect of the invention. In particular, the sample may be a blood sample, such as plasma or serum.

Suitable vessels include but are not limited medical tubes for collection of body fluid samples, such as blood collection tubes.

The sample preferably comprises a peptide, in particular a peptide as defined herein in the context of the first aspect of the invention.

In an embodiment, the vessel or device has a predefined filling volume, wherein the amount or concentration of the sulfone compound in the vessel is such that the concentration of the compound after addition of a sample in the predefined filing volume is at least 1% (v/v), in particular at least 1.25% (v/v), in particular at least 2% (v/v), in particular at least 2.5% (v/v) and in particular at least 5% (v/v). In embodiments, the concentration of the compound of the sulfone compound in the final incubation mixture (i.e. the sample comprising the peptide and the added sulfone compound) is at most 5% (v/v), in particular at most 10% (v/v) and in particular at most 20% (v/v).

In a sixth aspect, the present invention relates to a composition comprising a peptide (e.g. a gastrin peptide such as gastrin-17) and a sulfone compound (e.g. a compound of formula 3 as defined herein above, such as a compound of formula 1 or 2 as defined herein above.

What has been said elsewhere herein, especially any embodiments and definitions mentioned herein elsewhere with respect to other aspects (e.g. with respect to the first, second, third, fourth, and fifth aspect) applies to this sixth aspect of the invention mutatis mutandis.

The sulfone compound is a sulfone compound as defined in the embodiments of the first aspect of the invention.

In embodiments, the composition is a calibrator or control material for a method for detecting a peptide of interest. For example, the composition may comprise a predefined concentration of a gastrin peptide (e.g. gastrin-17) and a sulfone compound and may be a control or calibrator material for a method of detecting and/or quantifying a gastrin peptide (e.g. gastrin-17).

In embodiments, the composition further comprises a protease capable of cleaving the peptide. The protease may be selected from the group consisting of GluC, Chymotrypsin and/or Proline-endopeptidase, especially when the peptide is a gastrin peptide.

All embodiments with respect to proteases disclosed in the context of the first aspect of the invention apply mutatis mutandis.

The peptide may be any of the peptides as defined herein, in particular in the context of the first aspect of the invention. In a particular embodiment, the peptide is a gastrin peptide, in particular gastrin-17 or gastrin-34, in particular gastrin-17.

In embodiments, the composition also comprises a sample, e.g. a sample as defined herein elsewhere, e.g. in the context of the first aspect of the invention.

The concentration of the sulfone compound in the composition may be at at least 1% (v/v), in particular at least 1.25% (v/v), in particular at least 2% (v/v), in particular at least 2.5% (v/v) and in particular at least 5% (v/v). In embodiments, the concentration of the compound of the sulfone compound in the final incubation mixture (i.e. the sample comprising the peptide and the added sulfone compound) is at most 5% (v/v), in particular at most 10% (v/v) and in particular at most 20% (v/v).

The composition is (i) intended to be contacted with a protease or a protease-containing sample or (ii) comprises the protease. The protease is a protease capable of cleaving the peptide and is preferably selected from GluC, Chymotrypsin and/or Proline-endopeptidase. The peptide is preferably a gastrin peptide, even more preferably a gastrin-17 peptide. In a preferred embodiment, the composition also comprises a protease capable of cleaving the peptide. In a preferred embodiment, the protease is selected from GluC, Chymotrypsin and/or Proline-endopeptidase.

According to a seventh aspect, the present invention provides a kit, said kit comprising a sulfone compound, a composition of the sixth aspect of the invention and/or a vessel or device of the fifth aspect of the invention.

The sulfone compound may be any of the sulfone compounds as defined in the embodiments of the first aspect of the invention or herein elsewhere. For example, the sulfone compound may be a sulfone compound of Formula 3, in particular of Formula 2 or 1. What has been said elsewhere herein, especially any embodiments and definitions mentioned herein elsewhere with respect to other aspects (e.g. with respect to the first, second, third, fourth, fifth and/or sixth aspect) applies to this seventh aspect of the invention mutatis mutandis.

In embodiments the kit is a kit for (i) stabilizing a peptide (e.g. a gastrin peptide such as gastrin-17) in a sample (e.g. a body fluid such as blood), (ii) collecting a sample comprising a peptide, (iii) preprocessing a sample comprising a peptide for detection of the peptide or (iv) detecting a peptide in a sample.

In embodiments, the kit is for detecting the peptide (e.g. a gastrin peptide) in the sample and further comprises one or more reagents for detecting the peptide (e.g. one or more antibodies) In an eighth aspect, the present invention relates to the use of a sulfone compound for inhibiting a protease. Corresponding methods comprising such use are also provided.

Such methods may comprise adding the sulfone compound to a sample.

What has been said elsewhere herein e.g. with respect to the first, second, third, fourth, fifth, sixth and seventh aspect), especially any embodiments and definitions with respect to the sulfone compound, the proteases and the samples (applies to this eighth aspect of the invention mutatis mutandis.

In embodiments, the sulfone compound comprises less than 20 non-hydrogen atoms, even more preferably less than 10 non-hydrogen atoms. Non-hydrogen atoms means any chemical element other than hydrogen.

In another embodiment, the sulfone compound comprises less than 20 carbon atoms, even more preferably less than 10 carbon atoms.

In embodiments, the sulfone compound does not comprise any double and/or triple bonds between carbon atoms, i.e. does not comprise any carbon-carbon double and/or triple bonds.

In a specific embodiment, the sulfone compound does not comprise any double and triple bonds between carbon atoms and comprises less than 20 non-hydrogen atoms, even more preferably less than 10 non-hydrogen atoms.

In another specific embodiment, the sulfone compound does not comprise any double and triple bonds between carbon atoms and comprises less than 20 carbon atoms, even more preferably less than 10 carbon atoms.

In embodiments, the sulfone compound has a logP value of lower than 0.2. In embodiments, the suldone compound has a logP value of lower than −0.13 with an error of +/−0.30.

Sulfone compounds with correspondingly low logP values have the advantage of being suitably water soluble to be used in aqueous samples such as body fluids, in particular blood samples. As demonstrated in the appended Examples the sulfone compounds have calculated logP values in this range.

The partition coefficient, abbreviated P, is defined as a particular ratio of the concentrations of a solute between the two solvents (a biphase of liquid phases), specifically for un-ionized solutes, and the logarithm of the ratio is thus log P. When one of the solvents is water and the other is a non-polar solvent such as n-octanol, then the log P value is a measure of lipophilicity or hydrophobicity. The defined precedent is for the lipophilic and hydrophilic phase types to always be in the numerator and denominator respectively. In the context of the present invention the biphasic system for defining P and logP is n-octanol and water, i.e. the logP value is logP_{Octanol/water}. The logP value may be experimentally determined or calculated. Preferably the logP refers to the calculated logP value. The logP is preferably calculated using ACD/CehmSketch 2018.1 (File version C50H41).

In a specific embodiment, the sulfone compound is of formula 3.

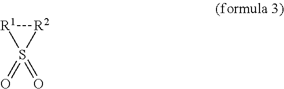

wherein R1 and R2 of formula 3 are either covalently linked via a R3 or not covalently linked,
wherein if R1 and R2 of formula 3 are covalently linked: i) R1 is selected from the group consisting of CH₂, CH—CH₃, NH and O, ii) R2 is selected from the group consisting of aliphatic linear or branched C1-C2, and iii) R3 is selected from the group consisting of aliphatic linear or branched C1-C4, CH—OR4 or C═O, wherein R4 is H or CH₃; or
wherein if R1 and R2 of formula 3 are not covalently linked: i) R1 is an aliphatic linear C1-C2, ii) R2 is an aliphatic linear C1-C2.

The dashed line between R1 and R2 in formula 3 indicates that these may either be covalently linked via R3 or not.

In embodiments, if R1 and R2 of formula 3 are covalently linked R 3 is selected from aliphatic linear or branched C1-C3, CH—OR4 or C═O, wherein R4 is H or CH₃.

In preferred embodiments, Formula 3 does not comprise any carbon-carbon double and triple bonds. In other words, any of the aliphatic structures mentioned in the definition of formula 3 above are saturated aliphatic structures.

In an even more specific embodiment, the sulfone compound is selected from formula 2 or formula 1.

Formula 1 refers to the following compound(s):

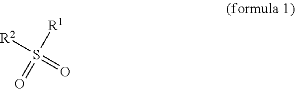

wherein R1 of formula 1 is an aliphatic linear C1-C2; and
wherein R2 of formula 1 is an aliphatic linear C1-C2.

In embodiments, R1 of formula 1 is a saturated aliphatic linear C1-C2; and R2 of formula 1 is a saturated aliphatic linear C1-C2.

In embodiments R1 of formula 1 is CH3-CH2 or CH3.

In embodiments R1 of formula 1 is methyl or ethyl.

In embodiments R2 of formula 1 is CH3-CH2 or CH3.

In embodiments R2 of formula 1 is methyl or ethyl.

In embodiments R1 of formula 1 is methyl and R2 of formula 1 is ethyl. In embodiments R1 of formula 1 is methyl and R2 of formula 1 is methyl. In embodiments R1 of formula 1 is ethyl and R2 of formula 1 is ethyl.

Formula 2 as used herein refers to the following compound(s):

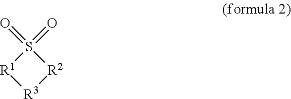

wherein R1 of formula 2 is selected from CH₂, CH—CH₃, NH and O,
wherein R2 of formula 2 is aliphatic linear or branched C1-C2,
wherein R3 of formula 2 is aliphatic linear or branched C1-C4, CH—OR4 or C═O, and wherein R4 is H or CH₃.

In embodiments, R1 of formula 2 is selected from CH₂, CH—CH₃, NH and O; R2 of formula 2 is saturated aliphatic linear or branched C1-C2; and R3 of formula 2 is saturated aliphatic linear or branched C1-C4, CH—OR4 or C═O, wherein R4 is H or CH₃.

In preferred embodiments R1 of formula 2 is CH₂, CH—CH₃ or O.

In preferred embodiments R2 of formula 2 is CH₂.

In preferred embodiments R3 of formula 2 is CH₂, CH₂—CH₂, CH₂—CHO—CH₂.

In specific embodiments, the sulfone compound is selected from the group consisting of: sulfolane (CAS: 126-33-0); trimethylene sulfone (CAS: 5687-92-3); 1,1-dioxoisothiazolidine (CAS: 5908-62-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4). As demonstrated in the appended Examples, all these compounds have the ability to stabilize peptides (e.g. gastrin-17) in samples (e.g. blood samples such as plasma or serum). Furthermore, all these substances are more stable against oxidation than DMSO.

In specific embodiments, the sulfone compound is selected from the group consisting of: sulfolane (CAS: 126-33-0); trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4). As demonstrated in the appended Examples, all these compounds had an ability to stabilize peptides (e.g. gastrin-17) in samples (e.g. blood samples such as plasma or serum) that is superior to the stabilizing ability of DMSO.

In specific embodiments, the sulfone compound is sulfolane (CAS: 126-33-0). Sulfolane is industrially produced in large amounts, i.e. is easily available in large amounts.

In specific embodiments, the sulfone compound is trimethylene sulfone (CAS: 5687-92-3).

In specific embodiments, the sulfone compound is 1,4-butanesultone (CAS: 1633-83-6).

In specific embodiments, the sulfone compound is tetrahydro-2H-thiopyran-4-ol 1,1-dioxide (CAS: 194152-05-1).

In specific embodiments, the sulfone compound is methyltetrahydrothiophene 1,1-dioxide (CAS: 1003-46-9).

In specific embodiments, the sulfone compound is ethylmethylsulfone (CAS: 594-43-4).

The protease may be selected from the group of EC 3.4 (group of peptide bond hydrolases) and the peptide is a cleavage target of such one or more protease. In an even more preferred embodiment, the protease is selected from the group of EC 3.4.21 (group of peptide bond hydrolases, which are serine proteases). In an even more preferred embodiment, the protease is selected from the group of: a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26).

“Inhibiting a protease” means that the presence of the compound at least slows down the degradation of a protease substrate, preferably in the context of a sample. A preferred substrate to test inhibition of the proteases is gastrin-17 (to the extent that this is a substrate of the proteases). Using gastrin.17 is in particular preferred in the context of GluC, Chymotrypsin and Proline-endopeptidase. Exemplary and preferred methods are disclosed in the appended Examples. Preferably, the stability in these assays is assessed at least for 24 h at the indicated temperatures.

The inhibition of a protease may take place in a sample, preferably a sample as defined in the embodiments of the first aspect of the invention. Such sample may comprise a peptide being a substrate of the protease which shall be stabilized.

The present invention in particular also relates to the following items:
1. Use (e.g. in vitro use) of a sulfone compound for stabilizing a peptide in a sample, optionally wherein the sulfone compound comprises less than 20 non-hydrogen atoms, even more preferably less than 10 non-hydrogen atoms, and/or optionally wherein the sulfone compound does not comprise any double and triple carbon-carbon bonds.
2. The use of item 1, wherein the sulfone compound has a logP value of lower than 0.2.
3. The use of item 1 or 2, wherein the sulfone compound stabilizes the peptide in the sample better than dimethylsulfoxid (DMSO; CAS: 67-68-5) after storage of at least 24 h at 20 °C.
4. The use of any one of items 1 to 3, wherein the sulfone compound is a compound of formula 3

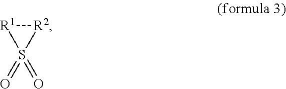

wherein R1 and R2 of formula 3 are either covalently linked via a R3 or not covalently linked (as indicated by the dashed line),
wherein if R1 and R2 of formula 3 are covalently linked: i) R1 is selected from the group consisting of CH₂, CH—CH₃, NH and O, ii) R2 is selected from the group consisting of aliphatic linear or branched C1-C2, and iii) R3 is selected from the group consisting of aliphatic linear or branched C1-C4, CH—OR4 or C═O, wherein R4 is H or CH₃; or
wherein if R1 and R2 of formula 3 are not covalently linked: i) R1 is an aliphatic linear C1-C2, ii) R2 is an aliphatic linear C1-C2.
5. The use of any one of items 1 to 4, wherein the sulfone compound is a compound of formula 2 or 1,
   wherein formula 2 is

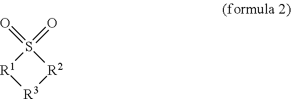

wherein R1 of formula 2 is selected from CH₂, CH—CH₃, NH and O,
wherein R2 of formula 2 is aliphatic linear or branched C1-C2,
wherein R3 of formula 2 is aliphatic linear or branched C1-C4, CH—OR4 or C═O,
wherein R4 is H or CH₃;
and
wherein formula 1 is

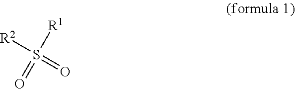

wherein R1 of formula 1 is an aliphatic linear C1-C2, and
wherein R2 of formula 1 is an aliphatic linear C1-C2.
6. The use of item 5, wherein R1 of formula 2 is CH₂, CH—CH₃ or O.
7. The use of item 5 or 6, wherein R2 of formula 2 is CH₂.
8. The use of any one of items 5 to 7, wherein R3 of formula 2 is CH₂, CH₂—CH₂, or CH₂—CHO—CH₂.
9. The use of any one of items 1 to 8, wherein the sulfone compound is selected from the group consisting of: Sulfolane; Trimethylene sulfone; 1,1-Dioxoisothiazolidine; 1,4-Butansultone; Tetrahydro-2H-thiopyran-4-ol 1,1-dioxide; Methyltetrahydrothiophene 1,1-dioxide; and Ethylmethylsulfone.
10. The use of any one of items 1 to 8, wherein the sulfone compound is selected from the group consisting of: Sulfolane; Trimethylene sulfone; 1,4-Butansultone; Tetrahydro-2H-thiopyran-4-ol 1,1-dioxide; Methyltetrahydrothiophene 1,1-dioxide; and Ethylmethylsulfone.
11. The use of item 5, wherein the sulfone compound is a compound of formula 1 as defined in item 5.
12. The use of item 5 or 11, wherein the sulfone compound of formula 1 is Ethylmethylsulfone.
13. The use of item 5, wherein the sulfone compound is a compound of formula 2.
14. The use of item 5 or 13, wherein the sulfone compound is selected from the group consisting of: Sulfolane; Trimethylene sulfone; 1,1-Dioxoisothiazolidine; 1,4-Butansultone; Tetrahydro-2H-thiopyran-4-ol 1,1-dioxide; Methyltetrahydrothiophene 1,1-dioxide.
15. The use of item 5 or 13, wherein the sulfone compound is selected from the group consisting of: Sulfolane; Trimethylene sulfone; 1,1-Dioxoisothiazolidine; 1,4-Butansultone; Tetrahydro-2H-thiopyran-4-ol 1,1-dioxide; and Methyltetrahydrothiophene 1,1-dioxide.
16. The use of any one of items 1 to 3, wherein the sulfone compound is Sulfolane.
17. The use of any one of items 1 to 3, wherein the sulfone compound is Trimethylene sulfone.
18. The use of any one of items 1 to 3, wherein the sulfone compound is 1,4-Butansultone.
19. The use of any one of items 1 to 3, wherein the sulfone compound is Tetrahydro-2H-thiopyran-4-ol 1,1-dioxide.
20. The use of any one of items 1 to 3, wherein the sulfone compound is Methyltetrahydrothiophene 1,1-dioxide.
21. The use of any one of items 1 to 3, wherein the sulfone compound is ethylmethylsulfone.
22. The use of any one of items 1 to 21, wherein the peptide has a length of 4 to 40 amino acids.
23. The use of any one of items 1 to 21, wherein the peptide has a length of 4 to 22 amino acids.
24. The use of any one of items 1 to 21, wherein the peptide has a length of 17 to 34 amino acids.
25. The use of any one of items 1 to 24, wherein the sample comprises one or more protease and the peptide comprises a recognition motif for said one or more protease.
26. The use of item 25, wherein the protease is a protease selected from the group of EC 3.4.
27. The use of item 25, wherein the protease is a protease selected from the group of EC 3.4.21.
28. The use of item 25, wherein the protease is a protease selected from the group consisting of a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26).
29. The use of item 25, wherein the one or more protease is selected from the group consisting of. GluC, Chymotrypsin and Proline-endopeptidase.
30. The use of any one of items 1 to 29, wherein the peptide is a gastrin peptide.
31. The use of item 30, wherein the gastrin peptide is selected from the group consisting of: a gastrin-17 peptide, a gastrin-34 peptide and a gastrin-14 peptide.
32. The use of item 30, wherein the gastrin peptide is a gastrin-17 peptide or a gastrin-34 peptide, in particular a gastrin-17 peptide, in particular mature gastrin-17.
33. The use of any one of items 1 to 32, wherein the peptide is stabilized from degradation, in particular proteolytic degradation.
34. The use of item 33, wherein the proteolytic degradation is a proteolytic degradation by one or more protease in the sample.
35. The use of item 34, wherein the protease is a protease selected from the group of EC 3.4.
36. The use of item 34, wherein the protease is a protease selected from the group of EC 3.4.21.
37. The use of item 34, wherein the protease is a protease selected from the group consisting of a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26).
38. The use of item 34, wherein the one or more protease is selected from the group consisting of GluC, Chymotrypsin and Proline-endopeptidase.
39. The use of any one of items 1 to 38, wherein the sample comprises a protease as defined in any one of items 35 to 38.
40. The use of any one of items 1 to 39, wherein the sample is a biological sample, such as a body fluid.
41. The use of any one of items 1 to 40, wherein sample is a blood sample, such as venous blood or capillary blood.
42. The use of any one of items 1 to 41, wherein the sample is whole blood, plasma or serum.
43. The use of any one of items 1 to 42, wherein the sample is plasma or serum, in particular plasma.
44. The use of any one of items 1 to 43, wherein the sample is an animal-derived sample, in particular a sample obtained from a mammalian, in particular a human or horse sample.
45. The use of any one of items 1 to 44, wherein the peptide is incubated in the sample in the presence of the sulfone compound at a temperature of −80 °C to 30 °C, in particular −20 °C to 28 °C and in particular 1 °C to 27 °C.
46. The use of any one of items 1 to 45, wherein the peptide is incubated in the sample in the presence of the sulfone compound for more than 5 hours, preferably more than 8 hours, preferably 23 h or most preferably more or 24 hours or more.
47. The use of any one of items 1 to 46 wherein the sulfone compound is present at a concentration of at least 1% (v/v), in particular at least 1.25% (v/v), in particular at least 2% (v/v), in particular at least 2.5% (v/v) and in particular at least 5% (v/v).
48. The use of any one of items 1 to 47, wherein the sulfone compound is present at a concentration of at most 10% (v/v).
49. The use of any one of items 1 to 48, wherein the use comprises adding the sulfone compound to the sample.
50. The use of any one of items 1 to 49, wherein the use comprises incubating the sample in the presence of the sulfone compound.
51. The use of any one of items 1 to 50, wherein the stabilization of the peptide is superior to the stabilization achieved with the same concentration of DMSO instead of the compound under the same conditions and incubation time, wherein the incubation time is 23 hours or more.
52. The use of any one of items 1 to 51, wherein the sulfone compound is not DMSO.
53. An in vitro method for stabilizing a peptide in a sample, said method comprising adding a sulfone compound to the sample comprising the peptide, optionally wherein the sulfone compound comprises less than 20 non-hydrogen atoms, even more preferably less than 10 non-hydrogen atoms, and/or optionally wherein the sulfone compound does not comprise any double and triple carbon-carbon bonds.
54. The method of item 53, wherein the sulfone compound has a logP value of lower than 0.2.
55. The method of item 53 or 54, wherein the sulfone compound stabilizes the peptide in the sample better than dimethylsulfoxid (DMSO; CAS: 67-68-5) after storage of at least 24 h at 20 °C.
56. The method of any one of items 53 to 55, wherein the sulfone compound is a compound of formula 3

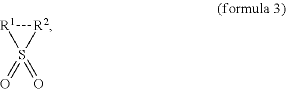

wherein R1 and R2 of formula 3 are either covalently linked via a R3 or not covalently linked (as indicated by the dashed line),
wherein if R1 and R2 of formula 3 are covalently linked: i) R1 is selected from the group consisting of CH₂, CH—CH₃, NH and O, ii) R2 is selected from the group consisting of aliphatic linear or branched C1-C2, and iii) R3 is selected from the group consisting of aliphatic linear or branched C1-C4, CH—OR4 or C═O, wherein R4 is H or CH₃; or
wherein if R1 and R2 of formula 3 are not covalently linked: i) R1 is an aliphatic linear C1-C2, ii) R2 is an aliphatic linear C1-C2.
57. The method of any one of items 53 to 56, wherein the sulfone compound is a compound of formula 2 or 1,
   wherein formula 2 is

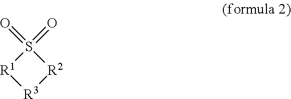

wherein R1 of formula 2 is selected from CH₂, CH—CH₃, NH and O,
wherein R2 of formula 2 is aliphatic linear or branched C1-C2,
wherein R3 of formula 2 is aliphatic linear or branched C1-C4, CH—OR4 or C═O,
wherein R4 is H or CH₃; and
wherein formula 1 is

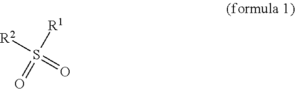

wherein R1 of formula 1 is an aliphatic linear C1-C2, and
wherein R2 of formula 1 is an aliphatic linear C1-C2.
58. The method of item 57, wherein R1 of formula 2 is CH₂, CH—CH₃ or O.
59. The method of item 57 or 58, wherein R2 of formula 2 is CH₂.
60. The method of any one of items 57 to 59, wherein R3 of formula 2 is CH₂, CH₂—CH₂, or CH₂—CHO—CH₂.
61. The method of any one of items 53 to 60, wherein the sulfone compound is selected from the group consisting of: Sulfolane; Trimethylene sulfone; 1,1-Dioxoisothiazolidine; 1,4-Butansultone; Tetrahydro-2H-thiopyran-4-ol 1,1-dioxide; Methyltetrahydrothiophene 1,1-dioxide; and Ethylmethylsulfone.
62. The method of any one of items 53 to 60, wherein the sulfone compound is selected from the group consisting of: Sulfolane; Trimethylene sulfone; 1,4-Butansultone; Tetrahydro-2H-thiopyran-4-ol 1,1-dioxide; Methyltetrahydrothiophene 1,1-dioxide; and Ethylmethylsulfone.
63. The method of item 57, wherein the sulfone compound is a compound of formula 1 as defined in item 57.
64. The method of item 57 or 63, wherein the sulfone compound of formula 1 is Ethylmethylsulfone.
65. The method of item 57, wherein the sulfone compound is a compound of formula 2.
66. The method of item 57 or 65, wherein the sulfone compound is selected from the group consisting of: Sulfolane; Trimethylene sulfone; 1,1-Dioxoisothiazolidine; 1,4-Butansultone; Tetrahydro-2H-thiopyran-4-ol 1,1-dioxide; Methyltetrahydrothiophene 1,1-dioxide.
67. The method of item 57 or 65, wherein the sulfone compound is selected from the group consisting of: Sulfolane; Trimethylene sulfone; 1,1-Dioxoisothiazolidine; 1,4-Butansultone; Tetrahydro-2H-thiopyran-4-ol 1,1-dioxide; and Methyltetrahydrothiophene 1,1-dioxide.
68. The method of any one of items 53 to 55, wherein the sulfone compound is Sulfolane.
69. The method of any one of items 53 to 55, wherein the sulfone compound is Trimethylene sulfone.
70. The method of any one of items 53 to 55, wherein the sulfone compound is 1,4-Butansultone.
71. The method of any one of items 53 to 55, wherein the sulfone compound is Tetrahydro-2H-thiopyran-4-ol 1,1-dioxide.
72. The method of any one of items 53 to 55, wherein the sulfone compound is Methyltetrahydrothiophene 1,1-dioxide.
73. The method of any one of items 53 to 55, wherein the sulfone compound is ethylmethylsulfone.
74. The method of any one of items 53 to 73, wherein the peptide has a length of 4 to amino acids.
75. The method of any one of items 53 to 74, wherein the peptide has a length of 4 to 22 amino acids.
76. The method of any one of items 53 to 75, wherein the peptide has a length of 17 to 34 amino acids.
77. The method of any one of items 53 to 76, wherein the sample comprises one or more protease and the peptide comprises a recognition motif for said one or more protease.
78. The method of item 77, wherein the protease is a protease selected from the group of EC 3.4.
79. The method of item 77, wherein the protease is a protease selected from the group of EC 3.4.21.
80. The method of item 77, wherein the protease is a protease selected from the group consisting of a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26).
81. The method of item 77, wherein the one or more protease is selected from the group consisting of: GluC, Chymotrypsin and Proline-endopeptidase.
82. The method of any one of items 1 to 81, wherein the peptide is a gastrin peptide.
83. The method of item 82, wherein the gastrin peptide is selected from the group consisting of: a gastrin-17 peptide, a gastrin-34 peptide and a gastrin-14 peptide.
84. The method of item 82, wherein the gastrin peptide is a gastrin-17 peptide or a gastrin-34 peptide, in particular a gastrin-17 peptide, in particular mature gastrin-17.
85. The method of any one of items 53 to 84, wherein the peptide is stabilized from degradation, in particular proteolytic degradation.
86. The method of item 85, wherein the proteolytic degradation is a proteolytic degradation by one or more protease in the sample.
87. The method of item 85, wherein the protease is a protease selected from the group of EC 3.4.
88. The method of item 85, wherein the protease is a protease selected from the group of EC 3.4.21.
89. The method of item 85, wherein the protease is a protease selected from the group consisting of a GluC protease (i.e. an enzyme of EC 3.4.21.19), a Chymotrypsin (i.e. an enzyme of EC 3.4.21.1) and a Proline-endopeptidase (i.e. an enzyme of EC 3.4.21.26).
90. The method of item 85, wherein the one or more protease is selected from the group consisting of GluC, Chymotrypsin and Proline-endopeptidase.
91. The method of any one of items 53 to 90, wherein the sample comprises a protease as defined in any one of items 35 to 38.
92. The method of any one of items 53 to 91, wherein the sample is a biological sample, such as a body fluid.
93. The method of any one of items 53 to 92, wherein sample is a blood sample, such as venous blood or capillary blood.
94. The method of any one of items 53 to 93, wherein the sample is whole blood, plasma or serum.
95. The method of any one of items 53 to 94, wherein the sample is plasma or serum, in particular plasma.
96. The method of any one of items 53 to 95, wherein the sample is an animal-derived sample, in particular a sample obtained from a mammalian, in particular a human or horse sample.
97. The method of any one of items 53 to 96, wherein the method further comprises incubating the peptide in the sample after adding the sulfone compound to the sample.
98. The method of item 97, wherein the incubation is at a temperature of −80 °C to 30 °C, in particular −20 °C to 28 °C and in particular 1 °C to 27 °C.
99. The method of item 97 or 98, wherein the incubation is for more than 5 hours, preferably more than 8 hours, preferably 23 h or most preferably more or 24 hours or more.
100. The method of any one of items 53 to 99 wherein the sulfone compound is added at a final concentration in the sample of at least 1% (v/v), in particular at least 1.25% (v/v), in particular at least 2% (v/v), in particular at least 2.5% (v/v) and in particular at least 5% (v/v).
101. The method of any one of items 53 to 100, wherein the compound is added at a final concentration in the sample of at most 10% (v/v).
102. The method of any one of items 53 to 101, wherein the stabilization of the peptide is superior to the stabilization achieved with the same concentration of DMSO instead of the compound under the same conditions and incubation time, wherein the incubation time is 23 hours or more.
103. The method of any one of items 53 to 102, wherein the sulfone compound is not DMSO.
104. An in vitro method for preprocessing a sample for the detection of a peptide in a sample, wherein said method comprises stabilizing the peptide according to a method as defined in any one of items 53 to 103.
105. An in vitro method for detecting a peptide in a sample, wherein the method comprises (i) stabilizing the peptide in the sample by a method as defined in any one of items 53 to 103 or preprocessing the sample by the method of item 104; and (ii) detecting the peptide in the sample.
106. A vessel for collecting a sample comprising a peptide (e.g. for the detection of the peptide), wherein said vessel comprises a sulfone compound, optionally wherein the sulfone compound comprises less than 20 non-hydrogen atoms, even more preferably less than 10 non-hydrogen atoms, and/or optionally wherein the sulfone compound does not comprise any double and triple carbon-carbon bonds.
107. The vessel of item 106, wherein the sulfone compound is a sulfone compound as defined in any one of items 2 to 21.
108. The vessel of item 106 or 107, wherein the sample is a sample as defined in any one of items 1 to 52.
109. The vessel of any one of items 106 to 108, wherein the peptide is a peptide as defined in any one of items 1 to 52.
110. The vessel of any one of items 106 to 109, wherein the vessel has a predefined filling volume range, wherein the amount or concentration of the compound in the vessel is such that the concentration of the compound after addition of the sample in the predefined filing volume range is as defined in item 47 and/or 48.
111. A composition comprising a peptide and a sulfone compound, optionally wherein the sulfone compound comprises less than 20 non-hydrogen atoms, even more preferably less than 10 non-hydrogen atoms, and/or optionally wherein the sulfone compound does not comprise any double and triple carbon-carbon bonds.
112. The composition of item 111 further comprising a protease capable of cleaving the peptide.
113. The composition of item 112, wherein the protease is as defined in any one of items to 38.
114. The composition of any one of items 111 to 113, wherein the peptide is a gastrin peptide, in particular gastrin-17 or gastrin-34, in particular gastrin-17.
115. The composition of any one of items 111 to 114, wherein the composition is a calibrator or control material for a method for detection of the peptide.
116. The composition of any one of items 111 to 115, wherein the peptide is a peptide as defined in any one of items 1 to 52.
117. The composition of any one of items 111 to 116, wherein the sulfone compound is a sulfone compound as defined in any one of items 2 to 21.
118. The composition of any one of items 111 to 117, wherein the composition comprises a sample, for example a sample as defined in any one of items 1 to 52.
119. The composition of any one of items 111 to 118, wherein the compound is not DMSO.
120. The composition of any one of items 111 to 119, wherein the concentration of the sulfone compound in the composition is as defined in item 47 and/or 48.
121. A kit for (i) stabilizing a peptide in a sample, (ii) collecting a sample comprising a peptide, (iii) preprocessing a sample comprising a peptide for detection of the peptide and/or (iv) detecting a peptide in a sample, said kit comprising a sulfone compound as defined in any one of items 1 to 21, a vessel as defined in any one of items 106 to 110 and/or a composition as defined in any one of items 111 to 120.
122. The kit of item 121, wherein the kit further comprises reagents for detecting the peptide.
123. Use of a sulfone compound for inhibiting a protease, optionally wherein the sulfone compound comprises less than 20 non-hydrogen atoms, even more preferably less than 10 non-hydrogen atoms, and/or optionally wherein the sulfone compound does not comprise any double and triple carbon-carbon bonds.
124. The use of item 123 wherein the sulfone compound is as defined in any one of items 2 to 21.
125. The use of item 123 or 124, wherein the protease is as a protease as defined in any one of items 35 to 38.

The following definitions and embodiments apply to all aspects and embodiments of the invention as well as the items above mutatis mutandis.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Also, as used herein, the singular terms “a”, “an,” and “the” include the plural reference unless the context clearly indicates otherwise.

Unless otherwise indicated, nucleic acids are written left to right in 5′ to 3′ orientation; amino acid sequences are written left to right in amino to carboxy orientation, respectively.

Features, integers, characteristics, compounds, chemical moieties or groups described in conjunction with a particular aspect, embodiment or example of the invention are to be understood to be applicable to any other aspect, embodiment or example described herein unless incompatible therewith.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper.

Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “in vitro” is used to indicate that a use, process or method is performed outside a living organism and preferably on body fluids, isolated tissues, organs or cells. An in vitro method or use may also be referred to an ex vivo method or use, respectively. The methods and uses disclosed herein are preferably in vitro methods and uses.

The term “sulfone compound” or sulfone as used herein relates to a class of structurally related chemical compounds, which are organosulfur compounds containing a sulfonyl group (i.e. a hexavalent sulfur atom which is double-bonded to each of two oxygen atoms). The sulfonyl group and the fact that these compounds are organic (i.e. comprise carbon atoms) are the characterizing features for sulfone compounds as referred to herein.

In a preferred embodiment, a sulfone compound of the invention is of formula 4

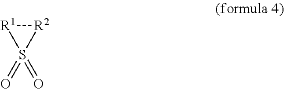

The dashed line in formula 4 indicates that R1 and R2 may or may not be covalently linked. In the event of a covalent linkage, this is not a direct bond but via a linker R3. R1 and/or R2 comprise at least one carbon atom.

In embodiments, the sulfone compound comprises less than 20 non-hydrogen atoms, even more preferably less than 10 non-hydrogen atoms. Non-hydrogen atoms means any chemical element other than hydrogen.

In another embodiment, the sulfone compound comprises less than 20 carbon atoms, even more preferably less than 10 carbon atoms.

In embodiments, the sulfone compound does not comprise any double and/or triple bonds between carbon atoms, i.e. does not comprise any carbon-carbon double and/or triple bonds. In embodiments, the sulfone compound does not comprise any non-saturated aliphatic structures.

In a specific embodiment, the sulfone compound does not comprise any double and triple bonds between carbon atoms and comprises less than 20 non-hydrogen atoms, even more preferably less than 10 non-hydrogen atoms.

The term peptide as used herein includes any molecules consisting of or essentially consisting of amino acids (e.g. naturally occurring amino acids). The term also includes modified peptides, e.g. peptides modified by non-amino acid components (e.g. chemical labels etc). A peptide as used herein can be of any length of amino acids and includes proteins (e.g. preprogastrin or progastrin). The terms peptide and protein are in principle used interchangeably herein. In embodiments, peptides comprise at most 40 amino acids and proteins comprise at least 41 amino acids.

Gastrin is a linear peptide hormone produced by G cells of the duodenum and in the pyloric antrum of the stomach of humans. It is secreted into the bloodstream. The encoded polypeptide is preprogastrin, which is cleaved by enzymes in posttranslational modification to produce progastrin (an intermediate, inactive precursor) and then gastrin in various forms, primarily the following three: gastrin-34, gastrin-17, gastrin-14. Also, pentagastrin is an artificially synthesized, five amino acid sequence identical to the last five amino acid sequence at the C-terminus end of gastrin. The numbers refer to the amino acid count.

The terms “gastrin peptide” or “gastrin peptides” as used herein relate to any peptide with a length of minimum 5 amino acids derived from preprogastrin (SEQ ID NO: 1). The sequences of preprogastrin and corresponding cleavage sites resulting in gastrin peptides are summarized in the UniProt entry P01350 (version 195). In particular, “gastrin peptide” or “gastrin peptides” include all naturally occurring gastrin peptides including but not limited to preprogastrin, progastrin, gastrin-34, gastrin-17, gastrin-14 and gastrin-6. In one embodiment, the gastrin peptide is selected from the group consisting of: preprogastrin, progastrin, gastrin-34, gastrin-17, gastrin-14. In a specific embodiment, the gastrin peptide is selected from the group consisting of: gastrin-34, gastrin-17.

Preprogastrin (SEQ ID NO: 1 shows human preprogastrin) is translated at the endoplasmic reticulum, where an N-terminal signaling sequence is cleaved from the peptide to yield progastrin. Progastrin (SEQ ID NO: 2 shows human Progastrin) then passes through the Golgi complex and the trans-Golgi network, where it can be sulfated on a tyrosine corresponding to the tyrosine residue in position 86 of preprogastrin (which becomes part of Gastrin-17 and Gastrin-34). The sulfation is not complete, i.e. gastrin peptides with and without sulfate on the Tyr corresponding to Tyr-86 or preprogastrin exist. Progastrin is further packaged into secretory vesicles, in which the dibasic sites corresponding to Arg36-Arg37 and Arg73-Arg74 are cleaved on the C-terminal side by the prohormone convertases PC1/3 and PC2, and the remaining C-terminal basic residues Arg73-Arg74 are removed by carboxypeptidase E to give the peptide gastrin-34-Gly (i.e. gastrin-34 with a C-terminal glycine). In the stomach, Gastrin-34-Gly is further processed by PAM enzyme to give the mature gastrin-34, which has a C-terminal amide group instead of the glycine. Mature gastrin-34 can be further processed to mature gastrin-17 by enzymatic cleavage and conversion of the N-terminal glutamine to pyro-glutamate. Alternatively, in the colon, gastrin-34-Gly can be processed to gastrin-17-Gly, i.e. having an N-terminal pyro-glutamate residue and a C-terminal glycine. The processing steps of preprogastrin including the different gastrin forms are for examples described in Copps J et al. (Protein Pept Lett. 2009; 16(12): 1504-1518. doi: 10.2174/092986609789839269).

The term gastrin-17 or gastrin-17 peptide as referred to herein relates to any types of glycin-17 peptides, i.e. gastrin-17-Gly, mature gastrin-17 (with C-terminal amide group instead of Gly in gastrin-17-Gly) both either sulfated or not sulfated at tyrosine in position 12 of the gastrin primary sequence. In other word, gastrin-17 as used herein includes and preferably relates to gastrin-17-Gly (SEQ ID NO: 3) and mature gastrin-17 (SEQ ID NO: 4). Both gastrin-17-Gly and mature gastrin-17 can be sulfated or not at the tyrosine at position 12. An optional further modification to any such forms of gastrin-17 are oxidation of the methionine at position 15, which can spontaneously occur, in particular in ex vivo samples.

Gastrin-34 or gastrin-34 peptide as used herein includes or preferably refers to gastrin 34-Gly (SEQ ID NO: 5) and mature gastrin-34 (with C-terminal amide group; SEQ ID NO: 6). Both gastrin-34-Gly and mature gastrin-34 can be sulfated or not at the tyrosine at position 29 (corresponding to position 12 of Gastrin-17). An optional further modification to any such forms of gastrin-34 are oxidation of the methionine at position 32 (corresponding to position 15 of gastrin-17), which can spontaneously occur especially ex vivo.

Gastrin-14 as used herein refers to a 14 amino acid peptide. The amino acid sequence of human gastrin-14 is depicted in SEQ ID NO: 7

The term “stabilizing a peptide” means that the peptide is degraded to a lesser extent than under corresponding control conditions. In the context of the compounds of the invention it specifically means that a peptide is degraded to a lower extent in the same period of time in a sample comprising the compound compared to the identical sample not comprising the compound. Corresponding assays for comparing the stabilization of peptides using no or different compounds (such as DMSO or compounds of the invention) are provided in the appended Examples. Preferably, at least 23 h are assessed using a gastrin peptide such as gastrin-17 as peptide.

“Recognition motif” of a protease as used herein relates to a sequence allowing recognition and cleavage by a corresponding protease. In other words, the presence of a recognition sequence for a protease in a peptide ensures that the peptide is a cleavage substrate for said protease under conditions at which the protease is active.

Description of Sequences
(Human Preprogastrin;
Uniprot: P013503, entry version 195)
SEQ ID NO: 1
MQRLCVYVLIFALALAAFSEASWKPRSQQPDAPLGTGANRDLELPWL
EQQGPASHHRRQLGPQGPPHLVADPSKKQGPWLEEEEEAYGWMDFGR
RSAEDEN
(Human Progastrin; Uniprot: P01350,
entry version 195, amino
acids 22 to 104)
SEQ ID NO: 2
SWKPRSQQPDAPLGTGANRDLELPWLEQQGPASHHRRQLGPQGPPHLVADPS
KKQGPWLEEEEEAYGWMDFGRRSAEDEN
(Human Gastrin-17-Gly)
SEQ ID NO: 3
XGPWLEEEEEAYGWMDFG,
wherein X is pyroglutamate
(Human Mature Gastrin-17)
SEQ ID NO: 4
XGPWLEEEEEAYGWMDF-NH2,
wherein X is pyroglutamate and NH2
represents an amide group.
(Human Gastrin-34-Gly)
SEQ ID NO: 5
XLGPQGPPHLVADPSKKQGPWLEEEEEAYGWMDFG,
wherein
X is pyroglutamate
(Human Mature Gastrin-34)
SEQ ID NO: 6
XLGPQGPPHLVADPSKKQGPWLEEEEEAYGWMDF-NH2,
wherein
X is pyroglutamate and NH2
represents an amide group
(Human Mature Gastrin-14)
SEQ ID NO: 7
WLEEEEEAYGWMDF-NH2,
wherein NH2 represents an amide group
Cleavage product gastrin-17
SEQ ID NO: 8
WLEEEEEAYGWMDF
Cleavage product gastrin-17
SEQ ID NO: 9
WLEEEEQAYGWMDF
Cleavage product gastrin-17
SEQ ID NO: 10
WLEEEEEAYGWM
Cleavage product gastrin-17
SEQ ID NO: 11
EEEEEAYGWMDF
Cleavage product gastrin-17
SEQ ID NO: 12
EEEEQAYGWMDF
Cleavage product gastrin-17
SEQ ID NO: 13
EEEEAYGWMDF
Cleavage product gastrin-17
SEQ ID NO: 14
EEEQAYGWMDF
Cleavage product gastrin-17
SEQ ID NO: 15
EEEAYGWMDF
Cleavage product gastrin-17
SEQ ID NO: 16
EEQAYGWMDF
Cleavage product gastrin-17
SEQ ID NO: 17
EEEEEAYGWM
Cleavage product gastrin-17
SEQ ID NO: 18
EEEEAYGWM
Cleavage product gastrin-17
SEQ ID NO: 19
EEEAYGWM

### DESCRIPTION OF THE FIGURES

The following figures are provided to aid the understanding of the present invention, the true scope of which is set forth in the appended claims, the aspects and embodiments as well as items described herein. It is understood that modifications can be made in the procedures set forth without departing from the spirit of the invention.

FIG. 1: Incubation of gastrin-17 in horse serum spiked (a) without additive; (b) with 5% [v/v] Sulfolane; (c) with 5% [v/v] DMSO. The MassSpec Peak area is plotted. Sulfolane shows better signal stability over time.

FIG. 2: Peptides A (SEQ ID NO: 8), B (SEQ ID NO: 9) and C (SEQ ID NO: 10) in horse serum spiked (a) without additive; (b) with 5% [v/v] Sulfolane; (c) with 5% [v/v] DMSO. The MassSpec Peak area is plotted. Sulfolane shows better signal stability over time.

FIG. 3: Peptide in horse serum spiked (a) with 5% [v/v] DMSO; (b) with 5% [v/v] Sulfolane; (c) with 2.5% [v/v] DMSO; (d) with 2.5% [v/v] Sulfolane; (e) with 1.25% [v/v] DMSO; (f) with 1.25% [v/v] Sulfolane; The MassSpec Peak area is plotted. 5% Sulfolane improves stability best.

FIG. 4: Gastrin-17 in horse serum spiked (a) with 5% [v/v]H₂O (b) with 5% [v/v] DMSO; (c) with 5% [v/v] Sulfolane; (d) with 5% [v/v] Trimethylene sulfone; (e) with 5% [v/v]1,1-Dioxoisothiazolidine; (f) with 5% [v/v]1,4-Butansulton; (g) with 5% [v/v] Tetrahydro-2H-thiopyran-4-ol 1,1-dioxide; (h) with 5% [v/v]methyltetrahydrothiophene 1,1-dioxide; (i) with 5% [v/v] Ethylmethylsulfone.

FIG. 5: UV-Chromatogram of Gastrin-17 in horse serum after 0 hours and 5 hours. The Gastrin-17 peak becomes smaller and many other peaks arise. Analysis of the degradation products using peptid mapping was done by proteome discoverer 2.5, Mascot, Static modification: Carbamidomethyl (C), pyro Glu, dynamic modification: Oxidation (M). Cause of gastrin-17 degradation are following enzymes: GluC, Chymotrypsin and Proline-endopeptidase, which are typical in blood serum, digestive tract or other body fluids of human and animals.

FIG. 6 shows the structures of DMSO (the reference compound) and the sulfone compounds used in the appended Examples and the corresponding CAS numbers.

### EXAMPLES

The following Examples are provided to aid the understanding of the present invention, the true scope of which is set forth in the appended claims, aspects and embodiments described herein. It is understood that modifications can be made in the procedures set forth without departing from the spirit of the invention.

### Example 1: Testing Different Substances (DMSO and Sulfolane) for their Ability to Stabilze Gastrin Peptides or Other Peptides

Methods:

Gastrin-17 (GAST_HUMAN(77-92)[Pyr-77]amid) and MS Qual/Quant QC-Mix (Mat. No.: MSQC2, Sigma Aldrich, a peptide mix comprising peptides A, B and C as referred to herein) was solved in horse serum with a concentration of 2.0 mg/ml and diluted to 0.22 mg/mL.

This solution was spiked respectively to reach a final peptide concentration of 0.1 mg/ml in (a) horse serum with added 5% [v/v]H₂O; (b) horse serum with added 5% [v/v] DMSO (CAS: 67-68-5); (c) horse serum with added 5% [v/v] Sulfolane (CAS: 126-33-0). The samples were stored at room temperature. Samples were taken at three times after spiking (0 h, 5 h and 23 h) and centrifuged with a Microcon-10 filter (Ultracel PL-10, Regenerated Cellulose 10,000 NMWL) for 10 min at 4 °C and 10,000 rpm. Afterwards the filtrate was separated by a liquid chromatography (HPLC) coupled with MS detector (QTOF Premier, Waters). The separation was done by a reverse phase liquid chromatography (Vydac 218TP C18, 5 μm, 2.1×150 mm, column temperature 50 °C) with a linear gradient to 100% of eluent B over 17 minutes. The eluent A was water with 0.025% [v/v]trifluoroacetic acid and eluent B was acetonitrile with 0.023% [v/v]trifluoroacetic acid. The quantification was done by using [M+H]⁺=1049.9 Da for Gastrin-17, [M+2H]²⁺=671.8 Da for peptid A, [M+2H]²⁺=751.4 Da for peptid B and [M+2H]²⁺=802.9 Da for peptid C. Peptid A, B and C are exemplary peptides contained in the peptid-Mix MS Qual/Quant QC-Mix (Mat. No.: MSQC2, Sigma Aldrich). The peptide mix MS Qual/Quant QC-Mix (Mat. No.: MSQC2, Sigma Aldrich) contained peptides of a length of 4 to 26 amino acids.

Results:

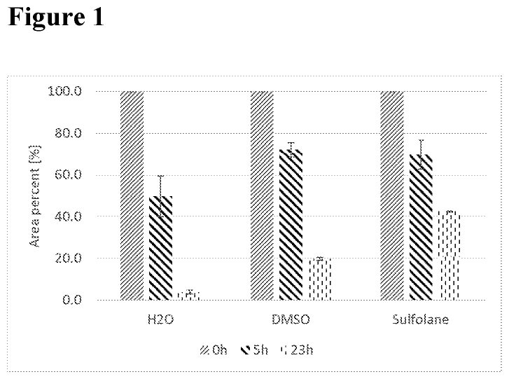

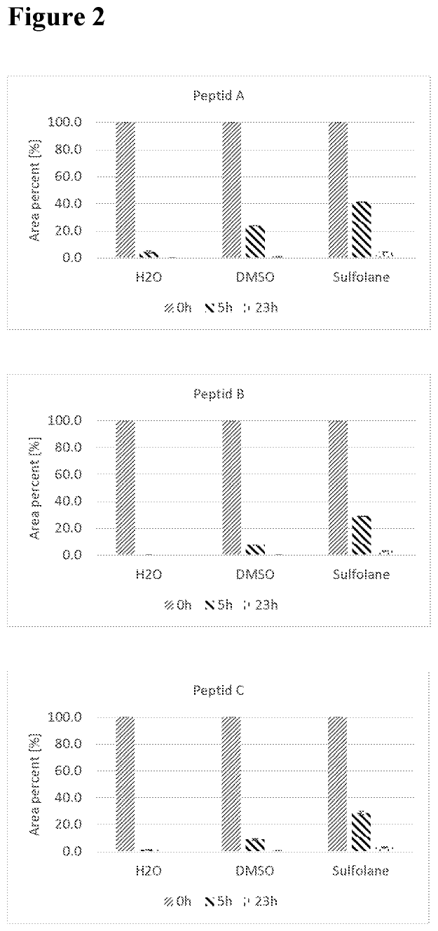

In this Example the ability of DMSO (CAS: 67-68-5) and Sulfolane (CAS: 126-33-0) to stabilize the gastrin peptide gastrin-17 and three peptides contained in a peptide mix (named peptides A, B and C) was compared. The results are shown in FIGS. 1 and 2, respectively.

### SUMMARY

Sulfolane and DMSO both exhibit protection ability on different peptides including the gastrin peptide gastrin-17. Sulfolane exceeds the DMSO protection outcome, especially after longer incubation (see 23 h).

### Example 2: Testing of Different Concentrations of DMSO and Sulfolane According to their Gastrin Protection Ability

Method:

Gastrin-17 (GAST_HUMAN(77-92)[Pyr-77]amid) and MS Qual/Quant QC-Mix (Mat. No.: MSQC2, Sigma Aldrich; comprises peptides A, B and C) was solved in horse serum with a concentration of 2.0 mg/ml and diluted to 0.22 mg/mL.

This solution was spiked respectively to reach a concentration of 0.1 mg/ml in (a) horse serum with added 5% [v/v] DMSO (CAS: 67-68-5); (b) horse serum with added 5% [v/v] Sulfolane (CAS: 126-33-0); c) horse serum with added 2.5% [v/v] DMSO (CAS: 67-68-5); d) horse serum with added 2.5% [v/v] Sulfolane (CAS: 126-33-0); e) horse serum with added 1.25% [v/v] DMSO (CAS: 67-68-5); f) horse serum with added 1.25% [v/v] Sulfolane (CAS: 126-33-0); The samples were stored at room temperature. A sample was taken 0 h and 5 h after spiking. The further experiment was conducted as described in Example 1.

Results:

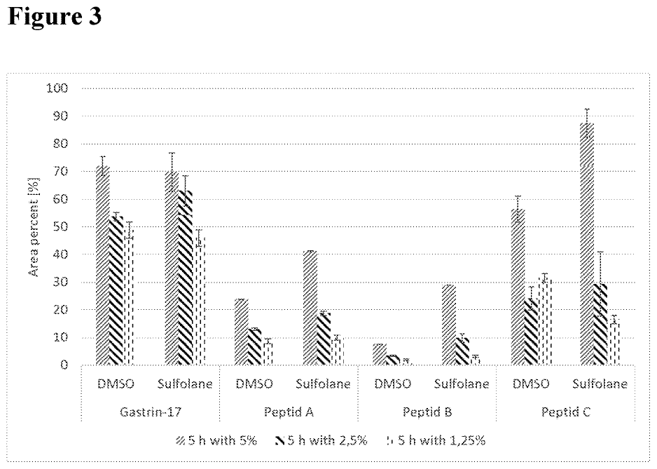

In this Example the ability of different concentrations of DMSO (CAS: 67-68-5) and Sulfolane (CAS: 126-33-0) to stabilize the gastrin peptide gastrin-17 and three peptides contained in a peptide mix (named peptides A, B and C) was compared. The results are shown in FIG. 3

Different concentrations of DMSO and Sulfolane exhibit different protection ability's but all tested concentrations 1.25% (V7V), 2.5% (V/V) and 5% (V/V) stabilized each of the peptides Gastrin-17, and peptides A, B and C. The stabilization increased with the concentration of DMSO or sulfolane used. Sulfolane showed an overall better peptide stabilization ability.

### Example 3: Testing Alternative and Structurally Similar Substances to Sulfolane (all Sulfone Compounds) for their Gastrin Protection Ability

Method:

Gastrin-17 (GAST_HUMAN(77-92)[Pyr-77]amid) was solved in horse serum with a concentration of 2.0 mg/ml and diluted to 0.22 mg/mL.

This solution was spiked respectively to reach a concentration of gastrin-17 of 0.1 mg/ml in (a) horse serum with added 5% [v/v]H₂O; (b) horse serum with added 5% [v/v] DMSO (CAS: 67-68-5); (c) horse serum added 5% [v/v] Sulfolane (CAS: 126-33-0); d) horse serum with added 5% [v/v]trimethylene sulfone (CAS: 5687-92-3); e) horse serum with added 5% [v/v]1,1-dioxoisothiazolidine (CAS: 5908-62-3); f) horse serum with added 5% [v/v]1,4-butanesultone (CAS: 1633-83-6); g) horse serum with added 5% [v/v]tetrahydro-2H-thiopyran-4-ol 1,1-dioxide (CAS: 194152-05-1); h) horse serum with added 5% [v/v]2-methyltetrahydrothiophene 1,1-dioxide (CAS: 1003-46-9); i) horse serum with added 5% [v/v]ethylmethylsulfone (CAS: 594-43-4). The samples were then stored/incubated at room temperature. Samples were taken after 0 h, 5 h and 24 h. The further experiment was performed as described in Example 1.

Results:

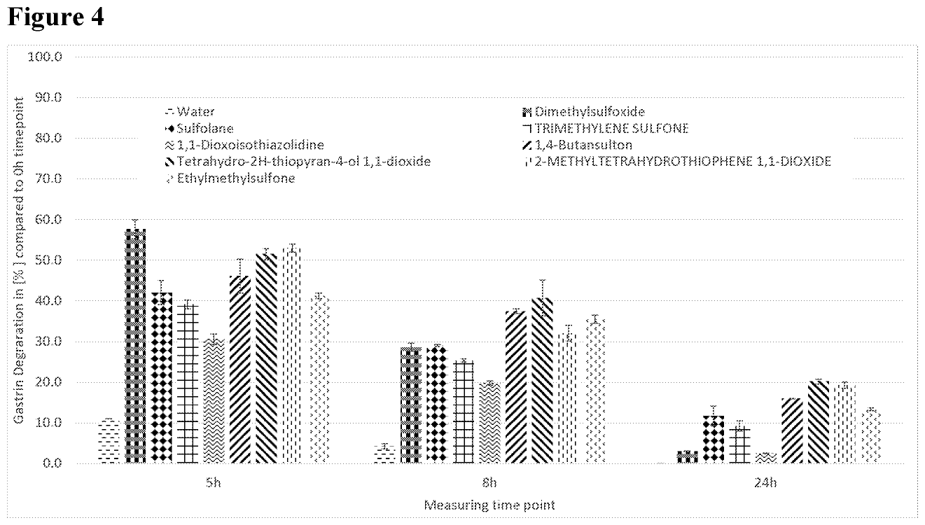

In this Example, the ability of different of compounds with a related structure to sulfolane and covered by either formula 1 or 2 to stabilize the gastrin peptide gastrin-17 peptide was assessed. The stabilization was assessed at 5 h and 24 h. The data is shown in FIG. 4 and demonstrates that all tested compounds have the ability to stabilize gastrin-17 in the serum samples. Surprisingly, not only sulfolane but at least also trimethylene sulfone (CAS: 5687-92-3); 1,4-butanesultone (CAS: 1633-83-6); tetrahydro-2H-thiopyran-4-ol 1,1-dioxide (CAS: 194152-05-1); methyltetrahydrothiophene 1,1-dioxide (CAS: 1003-46-9); and ethylmethylsulfone (CAS: 594-43-4) showed a stabilizing effect that is superior to the DMSO stabilizing effect.

### SUMMARY

The tested sulfone compounds with similar structures to Sulfolane covered by either formula 1 or 2 show all Gastrin protection ability.

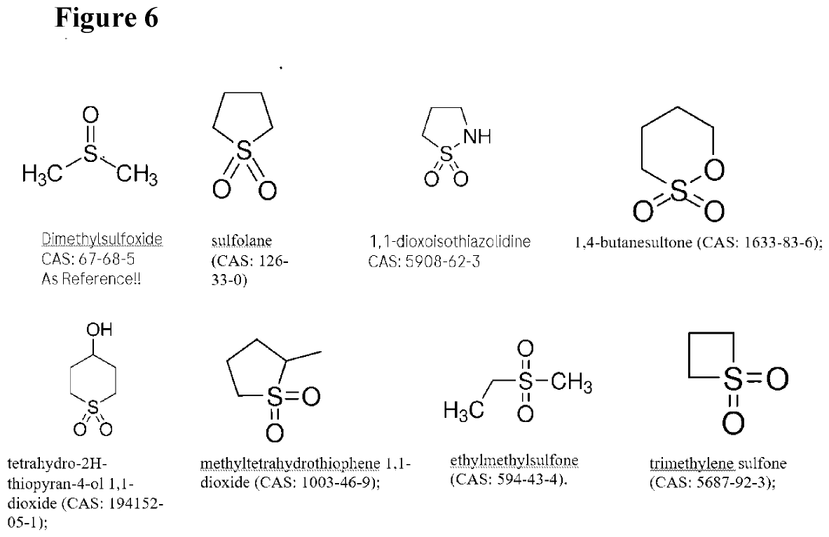

We found that Sulfolane and other sulfones (see FIG. 6) in similar concentrations like DMSO can protect the Gastrin Peptide better over a longer period (ca. 24 h) against serum derived degradation. Sulfolane and the other sulfones tested are fully water compatible substances (see table 1 below). Sulfolane is more stable than DMSO based upon oxidation.

TABLE 1
logP_{n-octanol/water} values calculated using ACD/CehmSketch
2018.1 (File version C50H41). The lower the logP
value the more water soluble is the compound.

| Sulfone Compound | Calculated logP_{n-octanol/water} |
|---|---|
| Sulfolane (CAS: 126-33-0) | −0.77(+/−0.39) |
| trimethylene sulfone (CAS: 5687-92-3) | −1.33 (+/−0.39) |
| 1,1-dioxoisothiazolidine (CAS: 5908-62-3) | −1.36 (+/−0.61) |
| 1,4-butanesultone (CAS: 1633-83-6) | −0.13 (+/−0.30) |
| tetrahydro-2H-thiopyran-4-ol 1,1-dioxide | −1.49 (+/−0.42) |
| (CAS: 194152-05-1) | |
| 2-methyltetrahydrothiophene 1,1-dioxide | −0.28 (+/−0.40) |
| (CAS: 1003-46-9) | |
| ethylmethylsulfone (CAS: 594-43-4) | −0.66 (+/−0.24) |

### Example 4: Determination of Gastrin-17 Degradation Products in Horse Serum and Identification of the Cleaving Proteases

Method:

Gastrin-17 (GAST_HUMAN(77-92)[Pyr-77]amid, BMO Nr.: 35.613602) was solved in horse serum with a concentration of 2.0 mg/ml and diluted to 0.1 mg/mL.

The gastrin-17 spiked serum was incubated at room temperature and samples were taken at two times (0 h and 5 h) and centrifuged with a Microcon-10 filter (Ultracel PL-10, Regenerated Cellulose 10,000 NMWL) for 10 min at 4 °C and 10,000 rpm. Afterwards the filtrate was separated by a liquid chromatography (HPLC) coupled with MS detector (Vanquish, Velos Orbitrap, Thermo Fisher Scientific). The separation was done by a reverse phase liquid chromatographie (X-Select CSH C18 Column, 130 Å, 3.5 μm, 2.1 mm×150 mm, P/N: 186005257, column temperature 50 °C) with a linear gradient to 30% of eluent B over 34 minutes and afterwards 6 min to 80% of eluent B. The entire chromatography took 45 min. The eluent A was water with 0.1% [v/v] formic acid and eluent B was acetonitrile with 0.1% [v/v] formic acid. Peptide mapping was done by proteome discoverer 2.5, Mascot, Static modification: Carbamidomethyl (C), pyro Glu, dynamic modification: Oxidation (M).

Results:

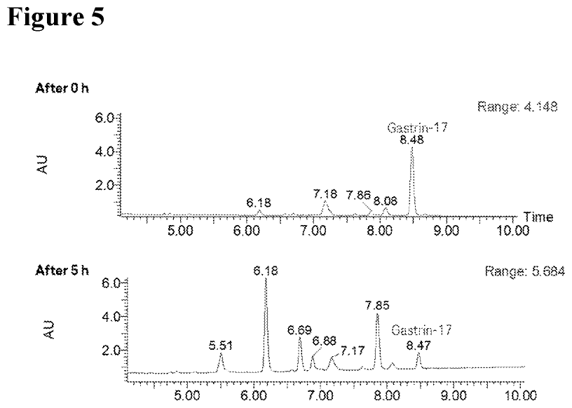

FIG. 5 depicts the UV chromatograms for the samples at 0 h and 5 h showing that the Gastrin-17 peak becomes smaller and many other peaks arise. This indicates that Gastrin-17 is proteolytically degraded in smaller peptides.

GC The peptides were identified as described above and are shown in Table 2 below.

[symbol]

[symbol]

indicates data missing or illegible when filed

1. (canceled)

2. An in vitro method for stabilizing a peptide in a sample, said method comprising adding a sulfone compound to the sample comprising the peptide, wherein the sulfone compound comprises less than 20 non-hydrogen atoms and wherein the sulfone compound does not comprise any carbon-carbon double and triple bonds.

3. The method of claim 2, wherein the sulfone compound has a logP value of lower than 0.2.

4. The method of claim 2, wherein the sulfone compound is a compound of formula 3 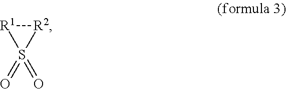
wherein R1 and R2 of formula 3 are either covalently linked via a R3 or not covalently linked (as indicated by the dashed line),
wherein if R1 and R2 of formula 3 are covalently linked: i) R1 is selected from the group consisting of CH₂, CH—CH₃, NIH and O, ii) R2 is an aliphatic linear or branched C1-C2, and iii) R3 is selected from the group consisting of an aliphatic linear or branched C1-C4, CH—OR4, and C═O, wherein R4 is H or CH₃; or
wherein if R1 and R2 of formula 3 are not covalently linked: i) R1 is an aliphatic linear C1-C2, ii) R2 is an aliphatic linear C1-C2.

5. The method of claim 2, wherein the sulfone compound is selected from the group consisting of: Sulfolane; Trimethylene sulfone; 1,4-Butansultone; Tetrahydro-2H-thiopyran-4-ol 1,1-dioxide; Methyltetrahydrothiophene 1,1-dioxide; and Ethylmethylsulfone.

6. The method of claim 2, wherein the sample comprises one or more protease and the peptide comprises a recognition motif for said one or more protease.

7. The se-er-method of claim 6, wherein the protease is a protease selected from the group consisting of a GluC protease (i.e. an enzyme of EC 3.4.21.19), a Chymotrypsin (i.e. an enzyme of EC 3.4.21.1), and a Proline-endopeptidase (i.e. an enzyme of EC 3.4.21.26).

8. The method of claim 2, wherein the peptide is a gastrin peptide.

9. An in vitro method for preprocessing a sample for the detection of a peptide in a sample, wherein said method comprises stabilizing the peptide according to a method as defined in claim 2.

10. An in vitro method for detecting a peptide in a sample, wherein the method comprises (i) stabilizing the peptide in the sample by a method as defined in claim 2; and (ii) detecting the peptide in the sample.

11. A device or vessel for collecting a sample, wherein said vessel comprises a sulfone compound, wherein the sulfone compound comprises less than 20 non-hydrogen atoms and wherein the sulfone compound does not comprise any carbon-carbon double and triple bonds.

12. The device or vessel of claim 11, wherein the sulfone compound is selected from the group consisting of Sulfolane: Trimethylene sulfone; 1,4-Butansultone, Tetrahydro-2H-thiopyran-4-ol 1,1-dioxide; Methyltetrahydrothiophene 1,1-dioxide; and Ethylmethylsulfone.

13. A composition comprising a peptide and a sulfone compound, wherein the sulfone compound comprises less than 20 non-hydrogen atoms and wherein the sulfone compound does not comprise any carbon-carbon double and triple bonds.

14. A kit for (i) stabilizing a peptide in a sample, (ii) collecting a sample comprising a peptide, (iii) preprocessing a sample comprising a peptide for detection of the peptide and/or (iv) detecting a peptide in a sample, said kit comprising a sulfone compound as defined in claim 2.

15. (canceled)

16. The method of claim 7, wherein the protease is a protease belonging to the group of EC 3.4.

17. The composition of claim 13, wherein the sulfone compound is as defined in claim 4.


SEQUENCE LISTING (19 sequences)

SEQ ID NO: 1 - protein, 101 aa, Homo sapiens
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN

SEQ ID NO: 2 - protein, 80 aa, Homo sapiens
SWKPRSQQPDAPLGTGANRDLELPWLEQQGPASHHRRQLGPQGPPHLVADPSKKQGPWLEEEEEAYGWMDFGRRSAEDEN

SEQ ID NO: 3 - protein, 18 aa, Homo sapiens
  SITE 1: note = X is pyroglutamate
XGPWLEEEEEAYGWMDFG

SEQ ID NO: 4 - protein, 18 aa, Homo sapiens
  SITE 1: note = X is pyroglutamate
  SITE 18: note = amide group (NH2)
XGPWLEEEEEAYGWMDFX

SEQ ID NO: 5 - protein, 35 aa, Homo sapiens
  SITE 1: note = X is pyroglutamate
XLGPQGPPHLVADPSKKQGPWLEEEEEAYGWMDFG

SEQ ID NO: 6 - protein, 35 aa, Homo sapiens
  SITE 1: note = X is pyroglutamate
  SITE 35: note = amide group (NH2)
XLGPQGPPHLVADPSKKQGPWLEEEEEAYGWMDFX

SEQ ID NO: 7 - protein, 15 aa, Homo sapiens
  SITE 15: note = X is amide group (NH2)
WLEEEEEAYGWMDFX

SEQ ID NO: 8 - protein, 14 aa, Homo sapiens
WLEEEEEAYGWMDF

SEQ ID NO: 9 - protein, 14 aa, Homo sapiens
WLEEEEQAYGWMDF

SEQ ID NO: 10 - protein, 12 aa, Homo sapiens
WLEEEEEAYGWM

SEQ ID NO: 11 - protein, 12 aa, Homo sapiens
EEEEEAYGWMDF

SEQ ID NO: 12 - protein, 12 aa, Homo sapiens
EEEEQAYGWMDF

SEQ ID NO: 13 - protein, 11 aa, Homo sapiens
EEEEAYGWMDF

SEQ ID NO: 14 - protein, 11 aa, Homo sapiens
EEEQAYGWMDF

SEQ ID NO: 15 - protein, 10 aa, Homo sapiens
EEEAYGWMDF

SEQ ID NO: 16 - protein, 10 aa, Homo sapiens
EEQAYGWMDF

SEQ ID NO: 17 - protein, 10 aa, Homo sapiens
EEEEEAYGWM

SEQ ID NO: 18 - protein, 9 aa, Homo sapiens
EEEEAYGWM

SEQ ID NO: 19 - protein, 8 aa, Homo sapiens
EEEAYGWM